SEQUENCE LISTING (63 sequences)

SEQ ID NO: 1 - DNA, 6774 bp, Homo sapiens
cggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtatagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctctttttcccaaaaggtaaaaatatagattgaaaagttaaaacattttgcatggctgcagttcctttgtttcttgagataagattccaaagaacttagattcatttcttcaacaccgaaatgctggaggtgtttgatcagttttcaagaaacttggaatataaataattttataattcaacaaaggttttcacattttataaggttgatttttcaattaaatgcaaatttgtgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctggaaagcttcacaattgtcacagacaaagatttttgttccaatactcgttttgcctctatttttcttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcattcgtctgccatttttcaattacatgctgacttcccttacaattgagatttgcccataggttaaacatggttagaaacaactgaaagcataaaagaaaaatctaggccgggtgcagtggctcatgcctatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttgcagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgtttgaaaataaattatggggatcctatttagctcttagtaccactaatcaaaagttcggcatgtagctcatgatctatgctgtttctatgtcgtggaagcaccggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagaacataggaaaaactaagaggttttgtttttatttttgctgatgaagagatatgtttaaatatgttgtattgttttgtttagttacaggacaataatgaaatggagtttatatttgttatttctattttgttatatttaataatagaattagattgaaataaaatataatgggaaataatctgcagaatgtgggttttcctggtgtttccctctgactctagtgcactgatgatctctgataaggctcagctgctttatagttctctggctaatgcagcagatactcttcctgccagtggtaatacgattttttaagaaggcagtttgtcaattttaatcttgtggatacctttatactcttagggtattattttatacaaaagccttgaggattgcattctattttctatatgaccctcttgatatttaaaaaacactatggataacaattcttcatttacctagtattatgaaagaatgaaggagttcaaacaaatgtgtttcccagttaactagggtttactgtttgagccaatataaatgtttaactgtttgtgatggcagtattcctaaagtacattgcatgttttcctaaatacagagtttaaataatttcagtaattcttagatgattcagcttcatcattaagaatatcttttgttttatgttgagttagaaatgccttcatatagacatagtctttcagacctctactgtcagttttcatttctagctgctttcagggttttatgaattttcaggcaaagctttaatttatactaagcttaggaagtatggctaatgccaacggcagtttttttcttcttaattccacatgactgaggcatatatgatctctgggtaggtgagttgttgtgacaaccacaagcactttttttttttttaaagaaaaaaaggtagtgaatttttaatcatctggactttaagaaggattctggagtatacttaggcctgaaattatatatatttggcttggaaatgtgtttttcttcaattacatctacaagtaagtacagctgaaattcagaggacccataagagttcacatgaaaaaaatcaatttatttgaaaaggcaagatgcaggagagaggaagccttgcaaacctgcagactgctttttgcccaatatagattgggtaaggctgcaaaacataagcttaattagctcacatgctctgctctcacgtggcaccagtggatagtgtgagagaattaggctgtagaacaaatggccttctctttcagcattcacaccactacaaaatcatcttttatatcaacagaagaataagcataaactaagcaaaaggtcaataagtacctgaaaccaagattggctagagatatatcttaatgcaatccattttctgatggattgttacgagttggctatataatgtatgtatggtattttgatttgtgtaaaagttttaaaaatcaagctttaagtacatggacatttttaaataaaatatttaaagacaatttagaaaattgccttaatatcattgttggctaaatagaataggggacatgcatattaaggaaaaggtcatggagaaataatattggtatcaaacaaatacattgatttgtcatgatacacattgaatttgatccaatagtttaaggaataggtaggaaaatttggtttctatttttcgatttcctgtaaatcagtgacataaataattcttagcttattttatatttccttgtcttaaatactgagctcagtaagttgtgttaggggattatttctcagttgagactttcttatatgacattttactatgttttgacttcctgactattaaaaataaatagtagatacaattttcataaagtgaagaattatataatcactgctttataactgactttattatatttatttcaaagttcatttaaaggctactattcatcctctgtgatggaatggtcaggaatttgttttctcatagtttaattccaacaacaatattagtcgtatccaaaataacctttaatgctaaactttactgatgtatatccaaagcttctcattttcagacagattaatccagaagcagtcataaacagaagaataggtggtatgttcctaatgatattatttctactaatggaataaactgtaatattagaaattatgctgctaattatatcagctctgaggtaatttctgaaatgttcagactcagtcggaacaaattggaaaatttaaatttttattcttagctataaagcaagaaagtaaacacattaatttcctcaacatttttaagccaattaaaaatataaaagatacacaccaatatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctattgtatgtgttaatgattttatgtaaaaatgtaattgcttttcatgagtagtatgaataaaattgattagtttgtgttttcttgtctccc

SEQ ID NO: 2 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - protein, 155 aa, Homo sapiens [sequence omitted]

SEQ ID NO: 4 - DNA, 695 bp, Mus musculus
ggccccgggccgttgtacactcaaggggctctctcggcttcaggaagagtccggctgcactgggctgggagcccggcgggacacggactgggaggctggcagcccgcgggcgagccgcgctggggggccgaggccggggtcggggccggggagccccaagagctgccacagcggggtcccggggccgcggaagggccatggctgccagcggcatcacctcgcttcccgcactgccggaggacggcggcgccgccttcccaccaggccacttcaaggaccccaagcggctctactgcaagaacggcggcttcttcctgcgcatccatcccgacggccgcgtggatggcgtccgcgagaagagcgacccacacgtcaaactacaactccaagcagaagagagaggagttgtgtctatcaagggagtgtgtgccaaccggtaccttgctatgaaggaagatggacggctgctggcttctaagtgtgttacagaagagtgtttcttctttgaacgactggaatctaataactacaatacttaccggtcacggaaatactccagttggtatgtggcactgaaacgaactgggcagtataaactcggatccaaaacgggacctggacagaaggccatactgtttcttccaatgtctgctaagagctgactcacttttgacactgtcactgagacactgtca

SEQ ID NO: 5 - protein, 154 aa, Mus musculus
MAASGITSLPALPEDGGAAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 6 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 7 - protein, 131 aa, Homo sapiens
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 8 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 9 - protein, 135 aa, Artificial Sequence
  Synthetic polypeptide
MRDSSPLDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 10 - protein, 132 aa, Artificial Sequence
  Synthetic polypeptide
GGQVDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 11 - protein, 128 aa, Artificial Sequence
  Synthetic polypeptide
DPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 12 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 13 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLVAEERGVVSIKGVCANRYLAMKEDGRLLASKSVTDECFFFERLESNAYNTYRSRKYTSWYVALKRTGQYKLGPKTGPGQKAILFLPMSAKS

SEQ ID NO: 14 - protein, 155 aa, Artificial Sequence
  Synthetic polypeptide
MAAGSITTLPALPEDGGSFAFPPGNYKDPVRLYCKNGGFFLRIHPDGRVDGVREKSDPHIKLQLQAEERGVVSIKGVCANRYLAMKEDGRLLASKCVTDECFFFERLESNNYVTYRSRKYTSWYVALKRTGQYKLGSKTGPGQKAILFLPMSAKS

SEQ ID NO: 15 - protein, 155 aa, Artificial Sequence
  Synthetic polypeptide
MAAGSITTLPALPEDGGSFAFPPGNYKDPKRLYCKNGGFFLRIHPDGRVDGVEEKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 16 - protein, 155 aa, Artificial Sequence
  Synthetic polypeptide
MAAGSITTLPALPEDGGSFAFPPGNYKDPKRLYCKNGGFFLRIHPDGRVDGVVEKSDPHIKLQLQAEERGVVSIKGVCANRYLAMKEDGRLLASKCVTDECFFFERLESNNYNTYRSRKYTSWYVALNRTGQYKLGSKTGPGQKAILFLPMSAKS

SEQ ID NO: 17 - protein, 155 aa, Artificial Sequence
  Synthetic polypeptide
MAAGSITTLPALPEDGGSFAFPPGNYKDPKRLYCKNGGFFLRIHPDGRVDGVVEKSDPHIKLQLQAEERGVVSIKGVCANRYLAMKEDGRLLASKCVTDECFFFERLESNNYNTYRSRKYTSWYVALDQTGQYVLGSKTGPGQKAILFLPMSAKS

SEQ ID NO: 18 - protein, 155 aa, Artificial Sequence
  Synthetic polypeptide
MAAGSITTLPALPEDGGSFAFPPGNYKDPVRLYCKNGGFFLRIHPDGRVDGVVEKSDPHIKLQLQAEERGVVSIKGVCANRYLAMKEDGRLLASKCVTDECFFFERLESNNYVTYRSRKYTSWYVALDQTGQYVLGSKTGPGQKAILFLPMSAKS

SEQ ID NO: 19 - protein, 155 aa, Artificial Sequence
  Synthetic polypeptide
MAAGSITTLPALPEDGGSFAFPPGNYKDPVRLYCKNGGFFLRIHPDGRVDGVREKSDPHIKLQLQAEERGVVSIKGVCANRYLAMKEDGRLLASKCVTDECFFFVRLESNNYNTYRSRKYTSWYVALKRTGQYKLGSKTGPGQKAILFLPMSAKS

SEQ ID NO: 20 - protein, 155 aa, Artificial Sequence
  Synthetic polypeptide
MAAGSITTLPALPEDGGSFAFPPGNYKDPVRLYCKNGGFFLRIHPDGRVDGVREKSDPHIKLQLQAEERGVVSIKGVCANRYLAMKEDGRLLASKCVTDECFFFERLESNNVNTYRSRKYTSWYVALKRTGQYKLGSKTGPGQKAILFLPMSAKS

SEQ ID NO: 21 - protein, 155 aa, Artificial Sequence
  Synthetic polypeptide
MAAGSITTLPALPEDGGSFAFPPGNYKDPVRLYCKNGGFFLRIHPDGRVDGVREKSDPHIKLQLQAEERGVVSIKGVCANRYLAMKEDGRLLASKCVTDECFFFERLESNNYVTYRSRKYTSWYVALKRTGRYKLGSKTGPGQKAILFLPMSAKS

SEQ ID NO: 22 - protein, 155 aa, Artificial Sequence
  Synthetic polypeptide
MAAGSITTLPALPEDGGSFAFPPGNYKDPVRLYCKNGGFFLRIHPDGRVDGVREKSDPHIKLQLQAEERGVVSIKGVCANRYLAMKEDGRLLASKCVTDECFFFVRLESNNYNTYRSRKYTSWYVALKRTGRYKLGSKTGPGQKAILFLPMSAKS

SEQ ID NO: 23 - protein, 155 aa, Artificial Sequence
  Synthetic polypeptide
MAAGSITTLPALPEDGGSFAFPPGNYKDPVRLYCKNGGFFLRIHPDGRVDGVREKSDPHIKLQLQAEERGVVSIKGVCANRYLAMKEDGRLLASKCVTDECFFFERLESNNVNTYRSRKYTSWYVALKRTGRYKLGSKTGPGQKAILFLPMSAKS

SEQ ID NO: 24 - protein, 135 aa, Artificial Sequence
  Synthetic polypeptide
MRDSSPLDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 25 - protein, 135 aa, Artificial Sequence
  Synthetic polypeptide
MRDSSPLDPKRLYCKNGGFFLRIHPDGRVDGVREKSDPHIKLQLVAEERGVVSIKGVCANRYLAMKEDGRLLASKSVTDECFFFERLESNAYNTYRSRKYTSWYVALKRTGQYKLGPKTGPGQKAILFLPMSAKS

SEQ ID NO: 26 - protein, 135 aa, Artificial Sequence
  Synthetic polypeptide
MRDSSPLDPVRLYCKNGGFFLRIHPDGRVDGVREKSDPHIKLQLQAEERGVVSIKGVCANRYLAMKEDGRLLASKCVTDECFFFERLESNNYVTYRSRKYTSWYVALKRTGQYKLGSKTGPGQKAILFLPMSAKS

SEQ ID NO: 27 - protein, 135 aa, Artificial Sequence
  Synthetic polypeptide
MRDSSPLDPKRLYCKNGGFFLRIHPDGRVDGVEEKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 28 - protein, 135 aa, Artificial Sequence
  Synthetic polypeptide
MRDSSPLDPKRLYCKNGGFFLRIHPDGRVDGVVEKSDPHIKLQLQAEERGVVSIKGVCANRYLAMKEDGRLLASKCVTDECFFFERLESNNYNTYRSRKYTSWYVALNRTGQYKLGSKTGPGQKAILFLPMSAKS

SEQ ID NO: 29 - protein, 135 aa, Artificial Sequence
  Synthetic polypeptide
MRDSSPLDPKRLYCKNGGFFLRIHPDGRVDGVVEKSDPHIKLQLQAEERGVVSIKGVCANRYLAMKEDGRLLASKCVTDECFFFERLESNNYNTYRSRKYTSWYVALDQTGQYVLGSKTGPGQKAILFLPMSAKS

SEQ ID NO: 30 - protein, 135 aa, Artificial Sequence
  Synthetic polypeptide
MRDSSPLDPVRLYCKNGGFFLRIHPDGRVDGVVEKSDPHIKLQLQAEERGVVSIKGVCANRYLAMKEDGRLLASKCVTDECFFFERLESNNYVTYRSRKYTSWYVALDQTGQYVLGSKTGPGQKAILFLPMSAKS

SEQ ID NO: 31 - protein, 135 aa, Artificial Sequence
  Synthetic polypeptide
MRDSSPLDPVRLYCKNGGFFLRIHPDGRVDGVREKSDPHIKLQLQAEERGVVSIKGVCANRYLAMKEDGRLLASKCVTDECFFFVRLESNNYNTYRSRKYTSWYVALKRTGQYKLGSKTGPGQKAILFLPMSAKS

SEQ ID NO: 32 - protein, 135 aa, Artificial Sequence
  Synthetic polypeptide
MRDSSPLDPVRLYCKNGGFFLRIHPDGRVDGVREKSDPHIKLQLQAEERGVVSIKGVCANRYLAMKEDGRLLASKCVTDECFFFERLESNNVNTYRSRKYTSWYVALKRTGQYKLGSKTGPGQKAILFLPMSAKS

SEQ ID NO: 33 - protein, 132 aa, Artificial Sequence
  Synthetic polypeptide
GGQVDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 34 - protein, 132 aa, Artificial Sequence
  Synthetic polypeptide
GGQVDPKRLYCKNGGFFLRIHPDGRVDGVREKSDPHIKLQLVAEERGVVSIKGVCANRYLAMKEDGRLLASKSVTDECFFFERLESNAYNTYRSRKYTSWYVALKRTGQYKLGPKTGPGQKAILFLPMSAKS

SEQ ID NO: 35 - protein, 132 aa, Artificial Sequence
  Synthetic polypeptide
GGQVDPVRLYCKNGGFFLRIHPDGRVDGVREKSDPHIKLQLQAEERGVVSIKGVCANRYLAMKEDGRLLASKCVTDECFFFERLESNNYVTYRSRKYTSWYVALKRTGQYKLGSKTGPGQKAILFLPMSAKS

SEQ ID NO: 36 - protein, 132 aa, Artificial Sequence
  Synthetic polypeptide
GGQVDPKRLYCKNGGFFLRIHPDGRVDGVEEKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 37 - protein, 132 aa, Artificial Sequence
  Synthetic polypeptide
GGQVDPKRLYCKNGGFFLRIHPDGRVDGVVEKSDPHIKLQLQAEERGVVSIKGVCANRYLAMKEDGRLLASKCVTDECFFFERLESNNYNTYRSRKYTSWYVALNRTGQYKLGSKTGPGQKAILFLPMSAKS

SEQ ID NO: 38 - protein, 132 aa, Artificial Sequence
  Synthetic polypeptide
GGQVDPKRLYCKNGGFFLRIHPDGRVDGVVEKSDPHIKLQLQAEERGVVSIKGVCANRYLAMKEDGRLLASKCVTDECFFFERLESNNYNTYRSRKYTSWYVALDQTGQYVLGSKTGPGQKAILFLPMSAKS

SEQ ID NO: 39 - protein, 132 aa, Artificial Sequence
  Synthetic polypeptide
GGQVDPVRLYCKNGGFFLRIHPDGRVDGVVEKSDPHIKLQLQAEERGVVSIKGVCANRYLAMKEDGRLLASKCVTDECFFFERLESNNYVTYRSRKYTSWYVALDQTGQYVLGSKTGPGQKAILFLPMSAKS

SEQ ID NO: 40 - protein, 132 aa, Artificial Sequence
  Synthetic polypeptide
GGQVDPVRLYCKNGGFFLRIHPDGRVDGVREKSDPHIKLQLQAEERGVVSIKGVCANRYLAMKEDGRLLASKCVTDECFFFVRLESNNYNTYRSRKYTSWYVALKRTGQYKLGSKTGPGQKAILFLPMSAKS

SEQ ID NO: 41 - protein, 132 aa, Artificial Sequence
  Synthetic polypeptide
GGQVDPVRLYCKNGGFFLRIHPDGRVDGVREKSDPHIKLQLQAEERGVVSIKGVCANRYLAMKEDGRLLASKCVTDECFFFERLESNNVNTYRSRKYTSWYVALKRTGQYKLGSKTGPGQKAILFLPMSAKS

SEQ ID NO: 42 - protein, 132 aa, Artificial Sequence
  Synthetic polypeptide
GGQVDPVRLYCKNGGFFLRIHPDGRVDGVREKSDPHIKLQLQAEERGVVSIKGVCANRYLAMKEDGRLLASKCVTDECFFFERLESNNYVTYRSRKYTSWYVALKRTGRYKLGSKTGPGQKAILFLPMSAKS

SEQ ID NO: 43 - protein, 132 aa, Artificial Sequence
  Synthetic polypeptide
GGQVDPVRLYCKNGGFFLRIHPDGRVDGVREKSDPHIKLQLQAEERGVVSIKGVCANRYLAMKEDGRLLASKCVTDECFFFVRLESNNYNTYRSRKYTSWYVALKRTGRYKLGSKTGPGQKAILFLPMSAKS

SEQ ID NO: 44 - protein, 132 aa, Artificial Sequence
  Synthetic polypeptide
GGQVDPVRLYCKNGGFFLRIHPDGRVDGVREKSDPHIKLQLQAEERGVVSIKGVCANRYLAMKEDGRLLASKCVTDECFFFERLESNNVNTYRSRKYTSWYVALKRTGRYKLGSKTGPGQKAILFLPMSAKS

SEQ ID NO: 45 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 46 - protein, 208 aa, Homo sapiens
MDSDETGFEHSGLWVSVLAGLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 47 - protein, 16 aa, Artificial Sequence
  Synthetic polypeptide
MRDSSPLVHYGWGDIP

SEQ ID NO: 48 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRARKYTSWYVALKRTGQYKLGSKTGPGQKAILFLPMSAKS

SEQ ID NO: 49 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYASRKYTSWYVALKRTGQYKLGSKTGPGQKAILFLPMSAKS

SEQ ID NO: 50 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSAKYTSWYVALKRTGQYKLGSKTGPGQKAILFLPMSAKS

SEQ ID NO: 51 - protein, 116 aa, Artificial Sequence
  Synthetic polypeptide
FLRIHPDGRVDGVREKSDPHIKLQLQAEERGVVSIKGVCANRYLAMKEDGRLLASKCVTDECFFFERLESNNYNTYRSRAYTSWYVALKRTGQYKLGSKTGPGQKAILFLPMSAKS

SEQ ID NO: 52 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSREYTSWYVALKRTGQYKLGSKTGPGQKAILFLPMSAKS

SEQ ID NO: 53 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSRKFTSWYVALKRTGQYKLGSKTGPGQKAILFLPMSAKS

SEQ ID NO: 54 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSRKYASWYVALKRTGQYKLGSKTGPGQKAILFLPMSAKS

SEQ ID NO: 55 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYAAAAFASWYVALKRTGQYKLGSKTGPGQKAILFLPMSAKS

SEQ ID NO: 56 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYAAAEFASWYVALKRTGQYKLGSKTGPGQKAILFLPMSAKS

SEQ ID NO: 57 - protein, 155 aa, Artificial Sequence
  Synthetic polypeptide
MAAGSITTLPALPEDGGSFAFPPGNYKDPKRLYCKNGGFFLRIHPDGAVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 58 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSRKATSWYVALKRTGQYKLGSKTGPGQKAILFLPMSAKS

SEQ ID NO: 59 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSRKYTSAYVALKRTGQYKLGSKTGPGQKAILFLPMSAKS

SEQ ID NO: 60 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSTKYTSWYVALKRTGQYKLGSKTGPGQKAILFLPMSAKS

SEQ ID NO: 61 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSRKYTAWYVALKRTGQYKLGSKTGPGQKAILFLPMSAKS

SEQ ID NO: 62 - protein, 155 aa, Artificial Sequence
  Synthetic polypeptide
MAAGSITTLPALPEDGGSFAFPPGNYKDPKRLYCKNGGFFLRIHPDGAVDGVREKSDPHIKLQLQAEERGVVSIKGVCANRYLAMKEDGRLLASKCVTDECFFFERLESNNYNTYRSTKAAAAYVALKRTGQYKLGSKTGPGQKAILFLPMSAKS

SEQ ID NO: 63 - protein, 155 aa, Artificial Sequence
  Synthetic polypeptide
MAAGSITTLPALPEDGGSFAFPPGNYKDPKRLYCKNGGFFLRIHPDGRVDGVREKSDPHIKLQLQAEERGVVYIKGVCANRYLAMKEDGRLLASKCVTDECFFFERLESNNYNTYRSRKYTSWYVALKRTGQYKLGSKTGPGQKAILFLPMSAKS

(19) United States
(12) Patent Application Publication
(10) Pub. No.: US 2018/0319857 A1
(43) Pub. Date: Nov. 8, 2018

(54) FGF2 TRUNCATIONS AND MUTANTS AND USES THEREOF

(71) Applicant: Salk Institute for Biological Studies, La Jolla, CA (US)

(72) Inventors: Ronald M. Evans, La Jolla, CA (US); Michael Downes, San Diego, CA (US); Annette Atkins, San Diego, CA (US); Ruth T. Yu, La Jolla, CA (US)

(73) Assignee: Salk Institute for Biological Studies, La Jolla, CA (US)

(21) Appl. No.: 16/034,999

(22) Filed: Jul. 13, 2018

Related U.S. Application Data
(63) Continuation of application No. PCT/US2017/014049, filed on Jan. 19, 2017.
(60) Provisional application No. 62/281,980, filed on Jan. 22, 2016.

(51) Int. Cl. C07K 14/50; A61P 3/10

(52) U.S. Cl. CPC C07K 14/50; A61P 3/10

(57) ABSTRACT

The present disclosure provides FGF2 mutant proteins, such as those having an N-terminal deletion, point mutation(s), or combinations thereof, which can reduce blood glucose in a mammal. Thus, the disclosed mutant FGF2 proteins can be used to treat one or more metabolic diseases. In some examples, mutant FGF2 proteins have reduced mitogenic activity. Also provided are nucleic acid molecules that encode such proteins, and vectors and cells that include such nucleic acids. Methods of using the disclosed molecules to reduce blood glucose levels, for example to treat a metabolic disorder are also provided.

### CROSS-REFERENCE TO RELATED APPLICATIONS

This application is a continuation of International Application No. PCT/US2017/014049, filed Jan. 19, 2017, which was published in English under PCT Article 21(2), which in turn which claims priority to U.S. Provisional Application No. 62/281,980 filed Jan. 22, 2016, both herein incorporated by reference.

### ACKNOWLEDGEMENT OF GOVERNMENT SUPPORT

This invention was made with government support under Grant Nos. DK057978, DK090962, HL088093, HL105278 and ES010337 awarded by The National Institutes of Health. The government has certain rights in the invention.

### FIELD

This application provides mutated FGF2 proteins, including FGF2 truncations, nucleic acids encoding such proteins, and methods of their use, for example to treat a metabolic disease, for example by reducing blood glucose levels.

### BACKGROUND

Type 2 diabetes and obesity are leading causes of mortality and are associated with the Western lifestyle, which is characterized by excessive nutritional intake and lack of exercise. A central player in the pathophysiology of these diseases is the nuclear hormone receptor (NHR) PPARγ, a lipid sensor and master regulator of adipogenesis. PPARγ is also the molecular target for the thiazolidinedione (TZD)-class of insulin sensitizers, which command a large share of the current oral anti-diabetic drug market. However, there are numerous side effects associated with the use of TZDs such as weight gain, liver toxicity, upper respiratory tract infection, headache, back pain, hyperglycemia, fatigue, sinusitis, diarrhea, hypoglycemia, mild to moderate edema, and anemia. Thus, the identification of new insulin sensitizers is needed.

### SUMMARY

Provided herein are mutants of fibroblast growth factor (FGF)2 that can be used to reduce blood glucose in a mammal, treat one or more metabolic diseases (e.g., one or more of diabetes, dyslipidemia, obesity, cardiovascular diseases, metabolic syndrome, and/or non alcoholic fatty liver disease (NAFLD)), or combinations thereof. In some examples, multiple metabolic diseases are treated simultaneously. In addition, methods of reducing fed and fasting blood glucose, improving insulin sensitivity and glucose tolerance, reducing systemic chronic inflammation, ameliorating hepatic steatosis in a mammal, or combinations thereof using the FGF2 mutant proteins (or nucleic acids encoding such) are provided herein. In some examples, use of the disclosed methods and FGF2 mutants result in one or more of: reduction in triglycerides, decrease in insulin resistance, reduction of hyperinsulinemia, increase in glucose tolerance, reduction of hyperglycemia, or combination thereof, in a mammal. Such FGF2 mutants can have an N-terminal truncation, point mutation(s), or combinations thereof. For example, FGF2 mutants can include mutations to reduce or even eliminate its mitogenic activity (such as a reduction of at least 20%, at least 30%, at least 40%, at least 50%, at least 75%, at least 80%, at least 90%, at least 95%, or even at least 99%, relative to a native/wild-type FGF2 protein). The disclosed FGF2 mutants can be used alone or in combination with other agents, such as other glucose reducing agents, such as thiazolidinedione.

Thus, provided herein are methods of reducing blood glucose, treating one or more metabolic diseases, or combinations thereof, in a mammal, which include administering a therapeutically effective amount of a mutated mature FGF2 protein to the mammal, or a nucleic acid molecule encoding the mutated mature FGF2 protein or a vector comprising the nucleic acid molecule, thereby reducing the blood glucose, treating the metabolic disease(s), or combinations thereof. Also provided are methods of reducing fed and fasting blood glucose, improving insulin sensitivity and glucose tolerance, reducing systemic chronic inflammation, ameliorating hepatic steatosis, or combinations thereof, in a mammal, which include administering a therapeutically effective amount of a mutated mature FGF2 protein to the mammal, or a nucleic acid molecule encoding the mutated FGF2 protein or a vector comprising the nucleic acid molecule.

Exemplary metabolic diseases that can be treated with the disclosed methods include but are not limited to: diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension). In some examples, one or more of these diseases are treated simultaneously with the disclosed FGF2 mutants.

In some examples, the mutated mature FGF2 protein includes deletion of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, or at least 27 contiguous N-terminal amino acids (such as 5, 6, 7, 8, 9, 10, 11, 12, 13, 14, 15, 16, 17, 18, 19, 20, 21, 22, 23, 24, 25, 26, or 27 contiguous N-terminal amino acids). In some examples, one or more of the deleted N-terminal amino acids are replaced with other amino acids, such as replacement of 1-10, 2-8 or 1, 2, 3, 4, 5, 6, 7, 8, 9, 10 replacements. For example, FGF2 can include alternative N-terminal sequences, such as those from FGF21 or an engineered tag shown to reduce the mitogenic activity of FGF19 (Zhou et al., Cancer Research, 2014, 74:3306-33016, herein incorporated by reference) (see FIG. 2) which can be used to alter the receptor binding specificity of the mutated FGF2 protein. In some examples, the mutated mature FGF2 protein includes at least one point mutation, such as a mutation at one or more of G19, H25, F26, K30, Y33, R53, Q65, S73, C96, E105, N111, Y112, N113, R116, S117, R118, K119, Y120, T121, S122, W123, K128, R129, Q132, K134, and S137 (such as a mutation at 1, 2, 3, 4, 5, 6, 7, 8, 9, 10, 11, 12, 13, 14, 15, 16, 17,18, 19, 20, 21, 22, 23, 24, 25, or all 26 of these positions), wherein the numbering refers to the sequence shown SEQ ID NO: 3. Specific exemplary point mutations are shown in Table 1. In some examples, the mutated FGF2 protein includes a combination of point mutation(s) and N-terminal deletions (e.g., see SEQ ID NOS: 9, 10, 24-44).

In some examples, the mutated mature FGF2 protein comprises at least 80%, at least 85%, at least 90%, at least 92%, at least 93%, at least 94%, at least 95%, at least 96%, at least 97%, at least 98%, or at least 99% sequence identity to the protein sequence shown in SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. For example, SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63 can have one or more, such as 1, 2, 3, 4, 5, 6, 7, 8, or 9 of the point mutations shown in Table 1, and/or one or more conservative amino acid substitutions (such as 1 to 20, 1 to 10, or 1 to 5 conservative substitutions). In some examples, the mutated mature FGF2 protein comprises or consists of the protein sequence shown in SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62 or 63.

Provided herein are mutated FGF2 proteins, which can include deletion of an N-terminal portion of FGF2, point mutations (such as amino acid substitutions, deletions, additions, or combinations thereof), or combinations of N-terminal deletions and point mutations, and methods of their use to lower glucose, treat one or more metabolic diseases, or combinations thereof (for example reduce fed and fasting blood glucose, improve insulin sensitivity and glucose tolerance, reduce systemic chronic inflammation, ameliorate hepatic steatosis in a mammal, or combinations thereof). In some examples, such mutations reduce the mitogenicity, such as a reduction of mitogenicity of at least 20%, at least 50%, at least 75% or at least 90% relative to a native mature FGF2 protein. In some examples, the mutant FGF2 protein is a truncated version of the mature protein (e.g., SEQ ID NO: 3), which can include for example deletion of at least 5, at least 6, at least 7, at least 8, at least 9, at least 10, at least 11, at least 12, at least 13, at least 14, at least 15, at least 16, at least 17, at least 18, at least 10, at least 20, at least 21, at least 22, at least 23, at least 24, at least 25, at least 26, or at least 27 contiguous N-terminal amino acids of mature FGF2. In some examples, the mutant FGF2 protein is a mutated version of the mature protein (e.g., SEQ ID NO: 3), such as one containing at least 1, at least 2, at least 3, at least 4, at least 5, at least 6, at least 7, at least 8, at least 9 or at least 10 amino acid substitutions (such as 1, 2, 3, 4, 5, 6, 7, 8, 9, 10, 11, 12, 13, 14, 15, 16, 17, 18, 19, 20, 21, 22, 23, 24, 25, or 26 substitutions), such as one or more of those shown in Table 1. In some examples, the mutant FGF2 protein includes both an N-terminal truncation and one or more point mutations. In some examples, the mutant FGF2 protein includes at least 20, at least 30, at least 40, or at least 50 consecutive amino acids of mature FGF2 (e.g., of SEQ ID NO:

3 or amino acids 10-154 of SEQ ID NO: 5), such as in the region of amino acids 46 to 95 of mature FGF2 (e.g., of SEQ ID NO: 3), (which in some examples can include 1-20 point mutations, such as substitutions, deletions, or additions). In some examples, the mutated mature FGF2 protein comprises at least 80%, at least 85%, at least 90%, at least 92%, at least 93%, at least 94%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. In some examples, the mutated mature FGF2 protein comprises or consists of the protein sequence shown in SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63.

Also provided are isolated nucleic acid molecules encoding the disclosed mutant FGF2 proteins. Vectors and cells that include such nucleic acid molecules are also provided.

The foregoing and other objects and features of the disclosure will become more apparent from the following detailed description, which proceeds with reference to the accompanying figures.

### BRIEF DESCRIPTION OF THE DRAWINGS

FIG. 1 shows an alignment of an exemplary mature form of FGF1 (SEQ ID NO: 6), a form of FGF1 with an N-terminal deletion (SEQ ID NO: 7), FGF2 (SEQ ID NO: 3), and an FGF2 sequence with 3 point mutations (G19F, H25N, F26Y) (SEQ ID NO: 8) referred to as FGF2 universal, with amino acids that form beta strands in bold, and other relevant residues highlighted and their interaction noted. FGF1 is referred to as the “universal” ligand as it can bind to all receptor subtypes.

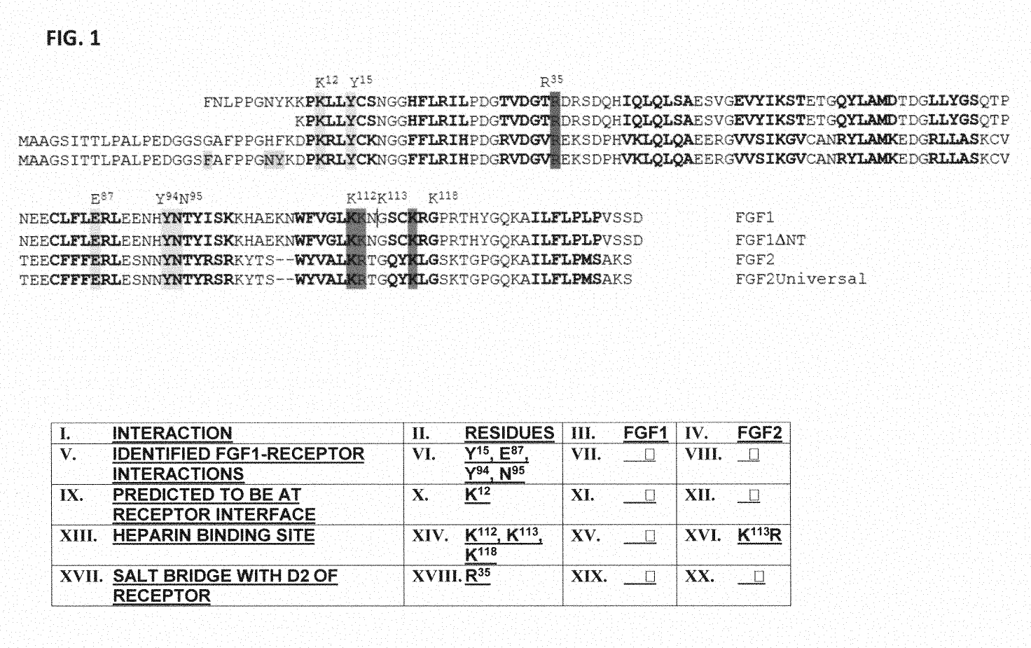

FIG. 2 shows an alignment of an exemplary human FGF2 sequence (SEQ ID NO: 3), a form of FGF2 with an N-terminal deletion and amino acids from the engineered FGF19 analog termed M70 (M70-FGF2; SEQ ID NO: 9) or from FGF21 (FGF21-FGF2; SEQ ID NO: 10).

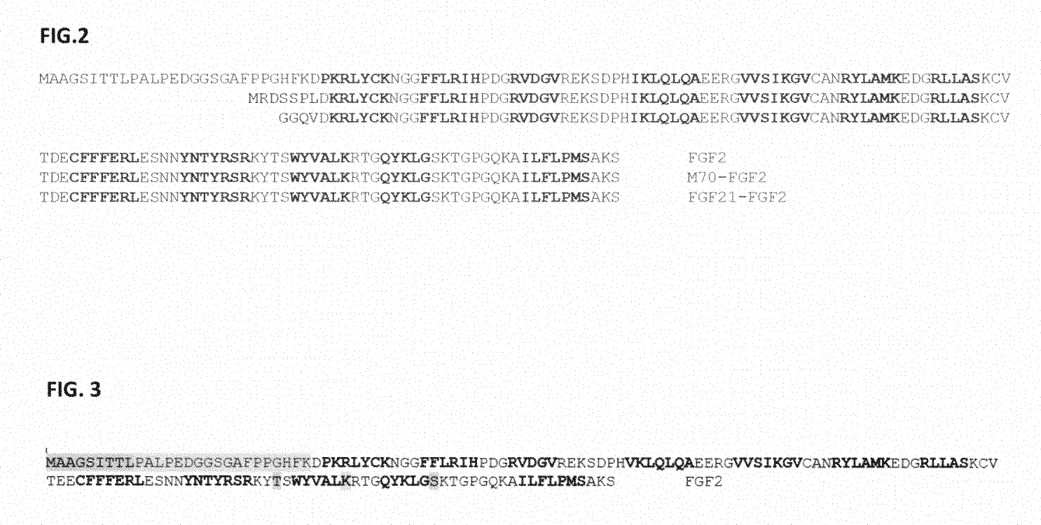

FIG. 3 shows an exemplary human FGF2 sequence (SEQ ID NO: 3), and highlights specific exemplary positions that can be mutated to improve thermal stability of the protein. For example, mutation K128N improves thermal stability. Introduction of mutations T121S, S137P, and deletion of the nine N-terminal amino acids improves thermal stability. In addition, mutations Q65L/I/V, N111A/G and C96S/T improve the thermal stability of the protein. Deletion of the first 27 N-terminal amino acids, FGF2 28-155 (SEQ ID NO: 11), in some examples is the maximum number of N-terminal deletions tolerated.

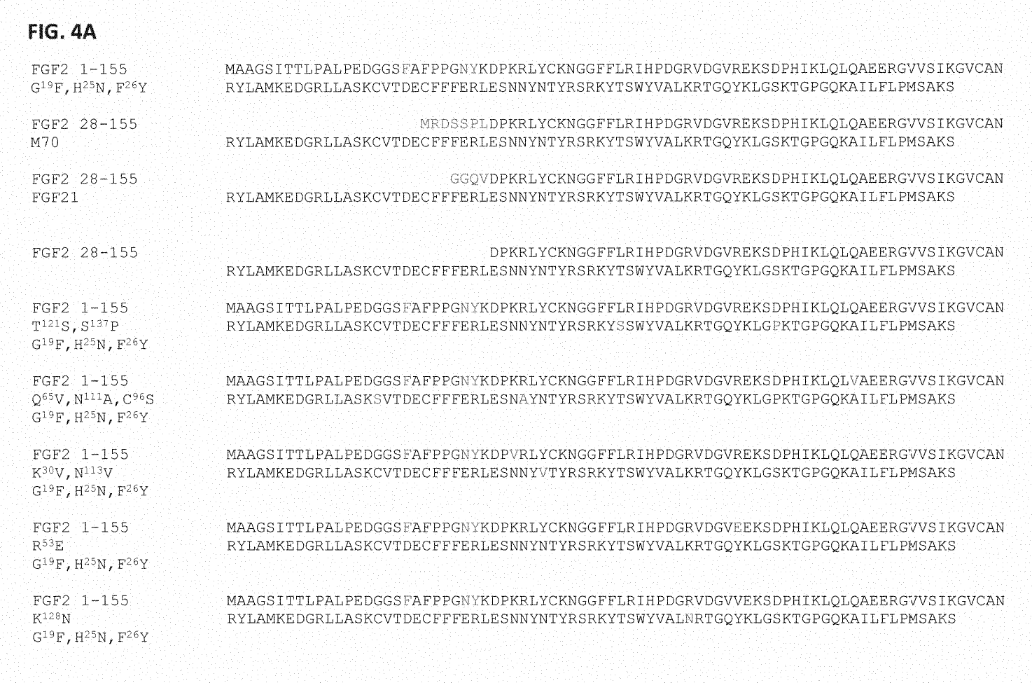

FIGS. 4A-4B show exemplary mutant FGF2 proteins that can be used in the methods provided herein. FGF2 (1-155aa) G19F, H25N, F26Y (SEQ ID NO: 8); FGF2 (28-155aa) M70 (SEQ ID NO: 9); FGF2 28-155 FGF21 (SEQ ID NO: 10); FGF2 28-15 (SEQ ID NO: 11); FGF2 1-155 T¹²¹S, S¹³⁷P, G¹⁹F, H²⁵N, F²⁶Y (SEQ ID NO: 12); FGF2 1-155 Q⁶⁵V,N¹¹¹A,C⁹⁶S, G¹⁹F,H²⁵N,F²⁶Y (SEQ ID NO: 13); FGF2 1-155 K³⁰V,N¹¹³V, G¹⁹F,H²⁵N,F²⁶Y (SEQ ID NO: 14); FGF2 1-155 R⁵³E, G¹⁹F,H²⁵N,F²⁶Y (SEQ ID NO: 15); FGF2 1-155 K¹²⁸N,G¹⁹F,H²⁵N,F²⁶Y (SEQ ID NO: 16); FGF2 1-155 K¹²⁸N, R¹²⁹Q,K¹³⁴V,G¹⁹F,H²⁵N,F²⁶Y (SEQ ID NO: 17); FGF2 1-155 K¹²⁸N, R¹²⁹Q,K¹³⁴V, K³⁰V,N¹¹³V,G¹⁹F,H²⁵N,F²⁶Y (SEQ ID NO: 18); FGF2 1-155 K³⁰V, E¹⁰⁵V,G¹⁹F,H²⁵N,F²⁶Y (SEQ ID NO: 19); FGF2 1-155 K³⁰V, Y¹¹²V,G¹⁹F,H²⁵N,F²⁶Y (SEQ ID NO: 20); FGF2 1-155 K³⁰V, N¹¹³V, Q¹³²R,G¹⁹F,H²⁵N,F²⁶Y (SEQ ID NO: 21); FGF2 1-155 K³⁰V, E¹⁰⁵V, Q¹³²R,G¹⁹F,H²⁵N,F²⁶Y (SEQ ID NO: 22); and FGF2 1-155 K³⁰V, Y¹¹²V, Q¹³²R,G¹⁹F,H²⁵N,F²⁶Y (SEQ ID NO: 23).

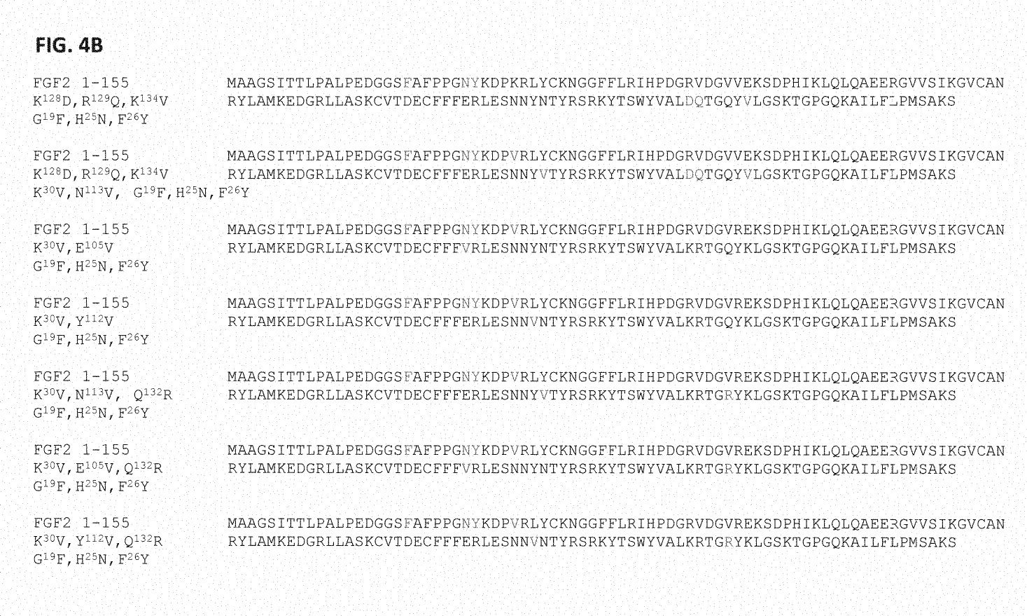

FIG. 5 shows exemplary mutant FGF2 proteins that incorporate the N-terminal residues introduced into the engineered FGF19 analog M70, which can be used in the methods provided herein: FGF2 28-155 M70 T¹²¹S,S¹³⁷P (SEQ ID NO: 24); FGF2 28-155 M70 Q⁶⁵V,N¹¹¹A,C⁹⁶S (SEQ ID NO: 25); FGF2 28-155 M70 K³⁰V,N¹¹³V (SEQ ID NO: 26); FGF2 28-155 M70 R⁵³E (SEQ ID NO: 27); FGF2 28-155 M70 K¹²⁸N (SEQ ID NO: 28); FGF2 28-155 M70 K¹²⁸D,R¹²⁹Q,K¹³⁴V (SEQ ID NO: 29); FGF2 28-155 M70 K¹²⁸D,R¹²⁹Q,K¹³⁴V,K³⁰V,N¹¹³V (SEQ ID NO: 30); FGF2 28-155 M70 K³⁰V,E¹⁰⁵V (SEQ ID NO: 31); and FGF2 28-155 M70 K³⁰V, Y¹¹²V (SEQ ID NO: 32).

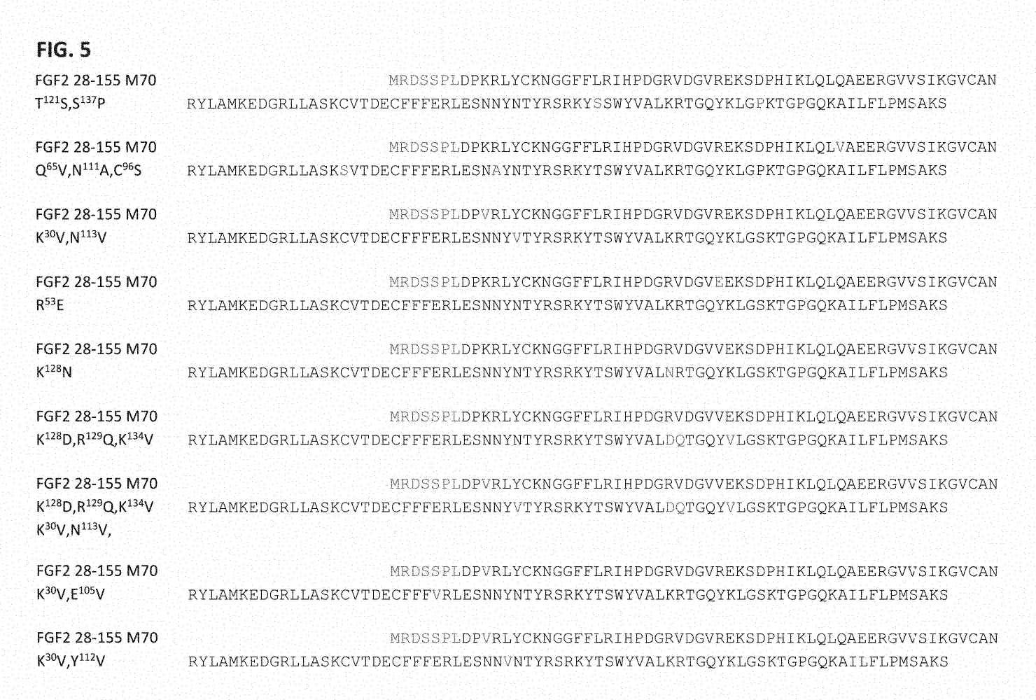

FIGS. 6A-6B show exemplary mutant FGF2-FGF21 proteins that can be used in the methods provided herein. FGF2 28-155 FGF21 T¹²¹S,S¹³⁷P (SEQ ID NO: 33); FGF2 28-155 FGF21 Q⁶⁵V,N¹¹¹A,C⁹⁶S (SEQ ID NO: 34); FGF2 28-155 FGF21 K³⁰V,N¹¹³V (SEQ ID NO: 35); FGF2 28-155 FGF21 R⁵³E (SEQ ID NO: 36); FGF2 28-155 FGF21 K¹²⁸N (SEQ ID NO: 37); FGF2 28-155 FGF21 K¹²⁸D,R¹²⁹Q,K¹³⁴V (SEQ ID NO: 38); FGF2 28-155 FGF21 K³⁰V,N¹¹³V,K¹²⁸D,R¹²⁹Q,K¹³⁴V (SEQ ID NO: 39); FGF2 28-155 FGF21 K³⁰V,E¹⁰⁵V (SEQ ID NO: 40); FGF2 28-155 FGF21 K³⁰V,Y¹¹²V (SEQ ID NO: 41); FGF2 28-155 FGF21 K³⁰V,N¹¹³V, Q¹³²R (SEQ ID NO: 42); FGF2 28-155 FGF21 K³⁰V,E¹⁰⁵V, Q¹³²R (SEQ ID NO: 43); and (SEQ ID NO: 44).

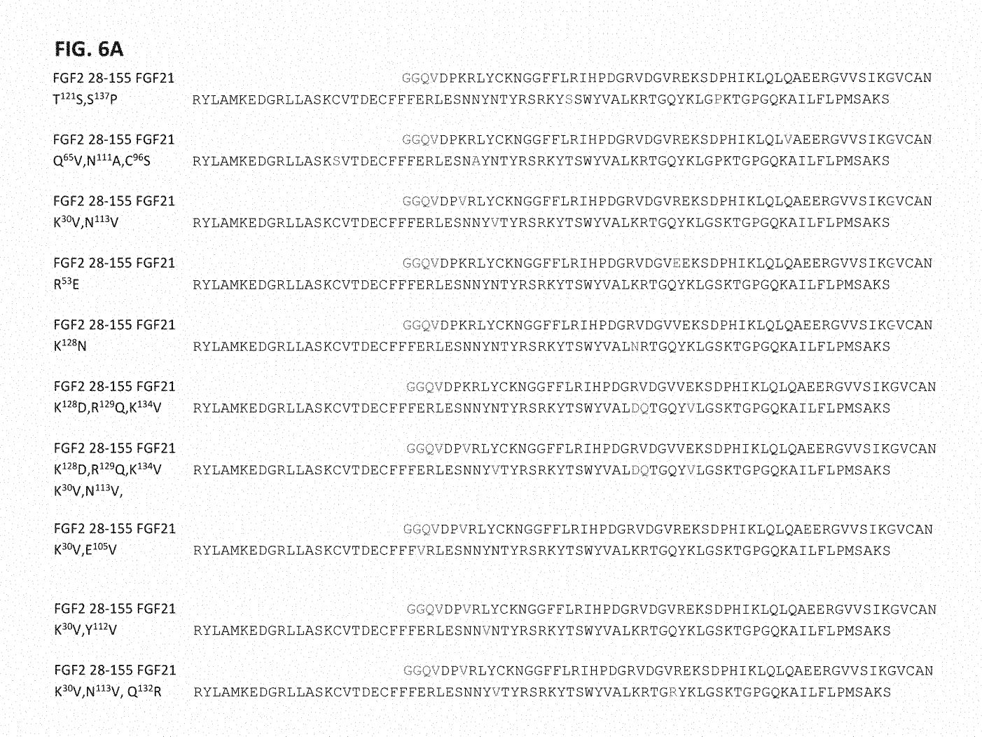

FIGS. 7A and 7B are bar graphs showing (A) blood glucose levels in ob/ob mice before (open bars) and 24 hours after (closed bars) a single subcutaneous injection of PBS (control), 0.5 mg/kg wild type human FGF1 (SEQ ID NO: 6), or 0.5 mg/kg wild type human FGF2 (SEQ ID NO: 3) and (B) the glucose lowering effects of mutFGF2 (SEQ ID NO: 8) in ob/ob mice. A single subcutaneous injection of mutFGF2 at 0.5 mg/kg was administered to ob/ob mice (n=6).

FIGS. 8A and 8B are a series of bar graphs showing the (A) glucose lowering effects or (B) food intake effects, in high fat diet fed wildtype and adipose-specific FGFR1 knockout mice. Mice were injected with vehicle, wild-type FGF2 (SEQ ID NO: 3), mutFGF2 (SEQ ID NO 8), or FGF1ΔNT (SEQ ID NO: 7).

FIGS. 9A and 9B are a series of bar graphs showing the (A) glucose lowering effects or (B) food intake effects, in high fat diet fed wildtype and adipose-specific FGFR2 knockout mice. Mice were injected with vehicle, wild-type FGF2 (SEQ ID NO: 3), mutFGF2 (SEQ ID NO 8), or FGF1ΔNT (SEQ ID NO: 7).

FIG. 10 provides an exemplary mutant FGF2 protein sequence with reduced mitogenicity. The sequence includes mutations G19F, H25N, F26Y, and S117A (SEQ ID NO: 48).

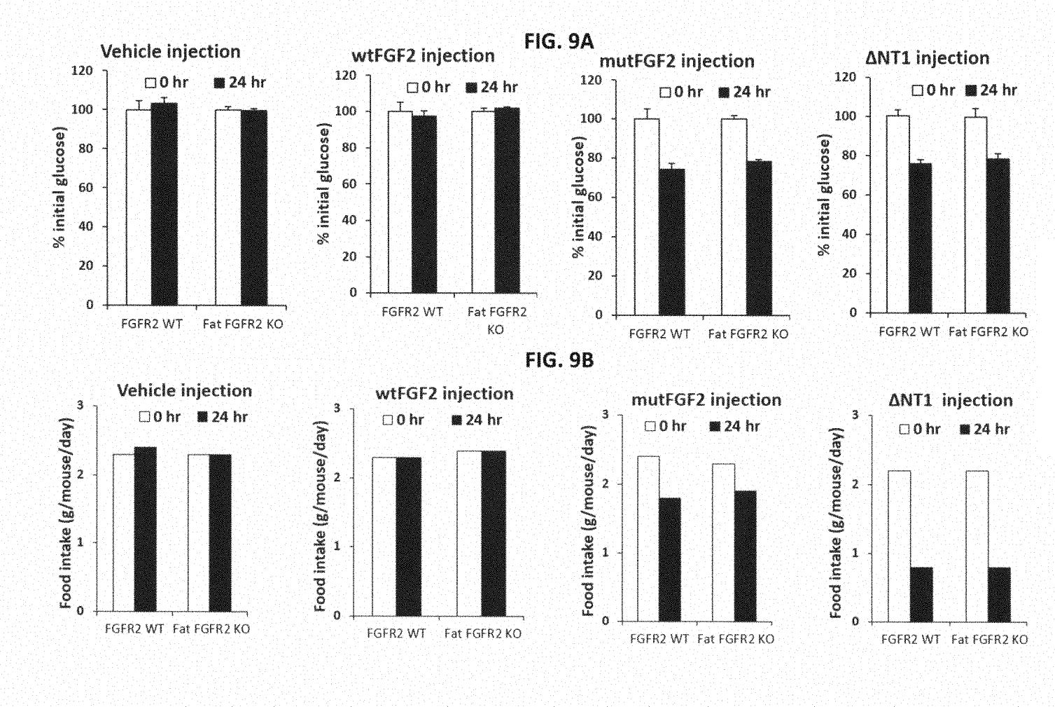

FIG. 11A provides an exemplary mutant FGF2 protein sequence (SEQ ID NO: 63).

FIG. 11B is a bar graph showing the ability of this sequence to lower blood glucose in vivo. The sequence includes mutations G19F, H25N, F26Y, and S73Y (SEQ ID NO: 63).

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Jan. 18, 2017, 9 k KB, which is incorporated by reference herein. In the accompanying sequence listing:

SEQ ID NOS: 1 and 2 provide an exemplary human FGF2 nucleic acid and protein sequences, respectively. Source: GenBank® Accession Nos: NM_002006.4 and NP_001997.5. FGF2 is alternatively translated from non AUG (amino acids 1 to 133 of SEQ ID NO: 2) and AUG initiation codons, resulting in five different isoforms. The AUG-initiated form is responsible for the paracrine effects. This 155 amino acid isoform of FGF2 (amino acids 134 to 288 of SEQ ID NO: 2) does not contain a signal sequence and is secreted by non-consensus pathways.

SEQ ID NO: 3 provides an exemplary mature form of a human FGF2 protein sequence. This is sometimes referred to as FGF2 (1-155aa). Underlined amino acids can be mutated (e.g., see Table 1).

| | |
|---|---|
| 1 | maagsittlp alpedggsga fppghfkdpk rlycknggff lrihpdgrvd gvreksdphi |
| 61 | klqlqaeerg vvsikgvcan rylamkedgr llaskcvtde cffferlesn nyntyrsrky |
| 121 | tswyvalkrt gqyklgsktg pgqkailflp msaks |

SEQ ID NOS: 4 and 5 provide an exemplary mouse FGF2 nucleic acid and protein sequences, respectively. Source: GenBank® Accession Nos: NM_008006.2 and NP_032032.1. FGF2 is alternatively translated from non AUG (amino acids 1 to 9 of SEQ ID NO: 5) and AUG initiation codons. The AUG-initiated form is responsible for the paracrine effects. This isoform of FGF2 does not contain a signal sequence and is secreted by non-consensus pathways.

SEQ ID NO: 6 provides an exemplary mature form of FGF1 (140 aa, sometimes referred to in the art as FGF1 15-154).

SEQ ID NO: 7 provides an exemplary mature form of FGF1 with an N-terminal truncation.

SEQ ID NO: 8 provides FGF2 (1-155αα) G19F, H25N, F26Y, an exemplary mature form of FGF2 with three point mutations (G19F, H25N, F26Y, wherein numbering refers to SEQ ID NO: 3) to reduce mitogenic activity.

SEQ ID NO: 9 provides an exemplary form of FGF2 with an N-terminal truncation, wherein some of the residues are replaced with those from an engineered FGF19 analog M70 selected for reduced mitogenicity (amino acids 1-7).

SEQ ID NO: 10 provides an exemplary form of FGF2 with an N-terminal truncation, wherein some of the residues are replaced with those from FGF21 (amino acids 1-4).

SEQ ID NO: 11 provides an exemplary form of FGF2, referred to as FGF2 28-155 with an N-terminal truncation of the first 27 N-terminal amino acids.

SEQ ID NO: 12 provides an exemplary mutated FGF2, referred to as FGF2 1-155 T¹²¹S, S¹³⁷P, G¹⁹F, H²⁵N, F²⁶Y with five point mutations (G¹⁹F, H²⁵N, F₂₆Y, T₁₂₁S, and S¹³⁷P).

SEQ ID NO: 13 provides an exemplary mutated FGF2, referred to as FGF2 1-155 Q⁶⁵V,N¹¹¹A,C⁹⁶S, G¹⁹F,H²⁵N,F²⁶Y with six point mutations (G¹⁹F, H²⁵N, F²⁶Y, Q⁶⁵V, N¹¹¹A, and C⁹⁶S).

SEQ ID NO: 14 provides an exemplary mutated FGF2, referred to as FGF2 1-155 K³⁰V N¹¹³V, G¹⁹F,H²⁵N,F²⁶Y with five point mutations (G¹⁹F, H²⁵N, F²⁶Y, K³⁰V and N¹¹³V).

SEQ ID NO: 15 provides an exemplary mutated FGF2, referred to as FGF2 1-155 R⁵³E, G¹⁹F,H²⁵N,F²⁶Y with four point mutations (G¹⁹F, H²⁵N, F²⁶Y and R⁵³E).

SEQ ID NO: 16 provides an exemplary mutated FGF2, referred to as FGF2 1-155 K¹²⁸N,G¹⁹F,H²⁵N,F²⁶Y with four point mutations (G¹⁹F, H²⁵N, F²⁶Y and K¹²⁸N).

SEQ ID NO: 17 provides an exemplary mutated FGF2, referred to as FGF2 1-155 K¹²⁸D, R¹²⁹Q,K¹³⁴V,G¹⁹F,H²⁵N,F²⁶Y with six point mutations (G¹⁹F, H²⁵N, F²⁶Y, K¹²⁸N, R¹²⁹Q, and K¹³⁴V).

SEQ ID NO: 18 provides an exemplary mutated FGF2, referred to as FGF2 1-155 K¹²⁸D, R¹²⁹Q,K¹³⁴V, K³⁰V,N¹¹³V,G¹⁹F,H²⁵N,F²⁶Y with eight point mutations (G¹⁹F, H²⁵N, F²⁶Y, K³⁰V, N¹¹³V, K¹²⁸N, R¹²⁹Q, and K¹³⁴V).

SEQ ID NO: 19 provides an exemplary mutated FGF2, referred to as FGF2 1-155 K³⁰V, E¹⁰⁵V,G¹⁹F,H²⁵N,F²⁶Y with five point mutations (G¹⁹F, H²⁵N, F²⁶Y, K³⁰V, and E¹⁰⁵V).

SEQ ID NO: 20 provides an exemplary mutated FGF2, referred to as FGF2 1-155 K³⁰V, Y¹¹²V,G¹⁹F,H²⁵N,F²⁶Y with five point mutations (G¹⁹F, H²⁵N, F²⁶Y, K³⁰V, and Y¹¹²V).

SEQ ID NO: 21 provides an exemplary mutated FGF2, referred to as FGF2 1-155 K³⁰V, N¹¹³V, Q¹³²R,G¹⁹F,H²⁵N,F²⁶Y with six point mutations (G¹⁹F, H²⁵N, F²⁶Y, K³⁰V, N¹¹³V, and Q¹³²R).

SEQ ID NO: 22 provides an exemplary mutated FGF2, referred to as FGF2 1-155 K³⁰V, E¹⁰⁵V, Q¹³²R,G¹⁹F,H²⁵N,F²⁶Y with six point mutations (G¹⁹F, H²⁵N, F²⁶Y, K³⁰V, E¹⁰⁵V, and Q¹³²R).

SEQ ID NO: 23 provides an exemplary mutated FGF2, referred to as FGF2 1-155 K30V, Y¹¹²V, Q¹³²R,G¹⁹F,H²⁵N,F²⁶Y with six point mutations (G¹⁹F, H²⁵N, F²⁶Y, K³⁰V, Y¹¹²V, and Q¹³²R).

SEQ ID NO: 24 provides an exemplary mutated FGF2, referred to as FGF2 28-155 M70 T¹²¹S,S¹³⁷P with a 27 amino acid N-terminal deletion and an N-terminal sequence derived from the non-mitogenic FGF19 analog referred to as M70 and two point mutations (T¹²¹S and S¹³⁷P).

SEQ ID NO: 25 provides an exemplary mutated FGF2, referred to as FGF2 28-155 M70 Q⁶⁵V,N¹¹¹A,C⁹⁶S with a 27 amino acid N-terminal deletion and an N-terminal sequence derived from the non-mitogenic FGF19 analog referred to as M70 and three point mutations (Q⁶⁵V, N¹¹¹A, and C⁹⁶S).

SEQ ID NO: 26 provides an exemplary mutated FGF2, referred to as FGF2 28-155 M70 K³⁰V,N¹¹³V with a 27 amino acid N-terminal deletion and an N-terminal sequence derived from the non-mitogenic FGF19 analog referred to as M70 and two point mutations (K³⁰V and N¹¹³V).

SEQ ID NO: 27 provides an exemplary mutated FGF2, referred to as FGF2 28-155 M70 R⁵³E with a 27 amino acid N-terminal deletion and an N-terminal sequence derived from the non-mitogenic FGF19 analog referred to as M70 and one point mutation (R⁵³E).

SEQ ID NO: 28 provides an exemplary mutated FGF2, referred to as FGF2 28-155 M70 K¹²⁸N with a 27 amino acid N-terminal deletion and an N-terminal sequence derived from the non-mitogenic FGF19 analog referred to as M70 and one point mutation (K¹²⁸N).

SEQ ID NO: 29 provides an exemplary mutated FGF2, referred to as FGF2 28-155 M70 K¹²⁸D,R¹²⁹Q,K¹³⁴V with a 27 amino acid N-terminal deletion and an N-terminal sequence derived from the non-mitogenic FGF19 analog referred to as M70 and three point mutations (K¹²⁸D, R¹²⁹Q, and K¹³⁴V).

SEQ ID NO: 30 provides an exemplary mutated FGF2, referred to as FGF2 28-155 M70 K¹²⁸D,R¹²⁹Q,K¹³⁴V,K³⁰V,N¹¹³V, with a 27 amino acid N-terminal deletion and an N-terminal sequence derived from the non-mitogenic FGF19 analog referred to as M70 and five point mutations (K³⁰V, N¹¹³V, K¹²⁸D, R¹²⁹Q, and K¹³⁴V).

SEQ ID NO: 31 provides an exemplary mutated FGF2, referred to as FGF2 28-155 M70 K³⁰V,E¹⁰⁵V with a 27 amino acid N-terminal deletion and an N-terminal sequence derived from the non-mitogenic FGF19 analog referred to as M70 and two point mutations (K³⁰V and E¹⁰⁵V).

SEQ ID NO: 32 provides an exemplary mutated FGF2, referred to as FGF2 28-155 M70 K³⁰V, Y¹¹²V with a 27 amino acid N-terminal deletion and an N-terminal sequence derived from the non-mitogenic FGF19 analog referred to as M70 and two point mutations (K³⁰V and Y¹¹²V).

SEQ ID NO: 33 provides an exemplary mutated FGF2, referred to as FGF2 28-155 FGF21 T¹²¹S,S¹³⁷P with a 27 amino acid N-terminal deletion and incorporating four N-terminal residues derived from the N-terminal sequence of FGF21 and two point mutations (T¹²¹S and S¹³⁷P).

SEQ ID NO: 34 provides an exemplary mutated FGF2, referred to as FGF2 28-155 FGF21 Q⁶⁵V,N¹¹¹A,C⁹⁶S with a 27 amino acid N-terminal deletion and incorporating four N-terminal residues derived from the N-terminal sequence of FGF21 and three point mutations (Q⁶⁵V, N¹¹¹A, C⁹⁶S).

SEQ ID NO: 35 provides an exemplary mutated FGF2, referred to as FGF2 28-155 FGF21 K³⁰V N¹¹³V with a 27 amino acid N-terminal deletion and incorporating four N-terminal residues derived from the N-terminal sequence of FGF21 and two point mutations (K³⁰V and N¹¹³V).

SEQ ID NO: 36 provides an exemplary mutated FGF2, referred to as FGF2 28-155 FGF21 R⁵³E with a 27 amino acid N-terminal deletion and incorporating four N-terminal residues derived from the N-terminal sequence of FGF21 and one point mutation (R⁵³E).

SEQ ID NO: 37 provides an exemplary mutated FGF2, referred to as FGF2 28-155 FGF21 K¹²⁸N with a 27 amino acid N-terminal deletion and incorporating four N-terminal residues derived from the N-terminal sequence of FGF21 and one point mutation (K¹²⁸N).

SEQ ID NO: 38 provides an exemplary mutated FGF2, referred to as FGF2 28-155 FGF21 K¹²⁸D,R¹²⁹Q,K¹³⁴V with a 27 amino acid N-terminal deletion and incorporating four N-terminal residues derived from the N-terminal sequence of FGF21 and three point mutations (K¹²⁸D, R¹²⁹Q, and K¹³⁴V).

SEQ ID NO: 39 provides an exemplary mutated FGF2, referred to as FGF2 28-155 FGF21 K³⁰V,N¹¹³V,K¹²⁸D,R¹²⁹Q,K¹³⁴V with a 27 amino acid N-terminal deletion and incorporating four N-terminal residues derived from the N-terminal sequence of FGF21 and five point mutations (K³⁰V, N¹¹³V, K¹²⁸D, R¹²⁹Q, and K¹³⁴V).

SEQ ID NO: 40 provides an exemplary mutated FGF2, referred to as FGF2 28-155 FGF21 K³⁰V,E¹⁰⁵V with a 27 amino acid N-terminal deletion and incorporating four N-terminal residues derived from the N-terminal sequence of FGF21 and two point mutations (K³⁰V and E¹⁰⁵V).

SEQ ID NO: 41 provides an exemplary mutated FGF2, referred to as FGF2 28-155 FGF21 K³⁰V,Y¹¹²V with a 27 amino acid N-terminal deletion and incorporating four N-terminal residues derived from the N-terminal sequence of FGF21 and two point mutations (K³⁰V and Y¹¹²V).

SEQ ID NO: 42 provides an exemplary mutated FGF2, referred to as FGF2 28-155 FGF21 K³⁰V,N¹¹³V, Q¹³²R with a 27 amino acid N-terminal deletion and incorporating four N-terminal residues derived from the N-terminal sequence of FGF21 and three point mutations (K³⁰V, N¹¹³V, and Q¹³²R).

SEQ ID NO: 43 provides an exemplary mutated FGF2, referred to as FGF2 28-155 FGF21 K³⁰V,E¹⁰⁵V, Q¹³²R with a 27 amino acid N-terminal deletion and incorporating four N-terminal residues derived from the N-terminal sequence of FGF21 and three point mutations (K³⁰V, E¹⁰⁵V, and Q¹³²R).

SEQ ID NO: 44 provides an exemplary mutated FGF2, referred to as FGF2 28-155 FGF21 K30V,Y¹¹²V, Q¹³²R with a 27 amino acid N-terminal deletion and incorporating four N-terminal residues derived from the N-terminal sequence of FGF21 and three point mutations (K³⁰V,Y¹¹²V, and Q¹³²R).

SEQ ID NO: 45 provides an exemplary human FGF19 protein sequence. Source: GenBank Accession No: NP_005108.1. The signal peptide is amino acids 1-22 (nt 454-529), and the mature FGF19 peptide is amino acids 23-216 (encoded by nt 530-1111). The mature form of FGF19 is sometimes referred to as FGF19 (23-216αα).

SEQ ID NO: 46 provides an exemplary human FGF21 protein sequence. Obtained from GenBank Accession No. AAQ89444.1. The mature form of FGF21 is about amino acids 21-208.

SEQ ID NO: 47 provides an exemplary modified portion of FGF19 with three amino acid substitutions (A30S, G31S, and H33L), referred to as M70.

SEQ ID NO: 48 provides an exemplary mutant FGF2 protein sequence. The sequence includes mutations G19F, H25N, F26Y, and S117A. The equivalent aa of S117 in FGF2 is S100 in FGF1.

SEQ ID NO: 49 provides an exemplary mutant FGF2 protein sequence. The sequence includes mutations G19F, H25N, F26Y, and R116A.

SEQ ID NO: 50 provides an exemplary mutant FGF2 protein sequence. The sequence includes mutations G19F, H25N, F26Y, and R118A. SEQ ID NO: 51 provides an exemplary mutant FGF2 protein sequence. The sequence includes mutations G19F, H25N, F26Y, and K119A.

SEQ ID NO: 52 provides an exemplary mutant FGF2 protein sequence. The sequence includes mutations G19F, H25N, F26Y, and K119E.

SEQ ID NO: 53 provides an exemplary mutant FGF2 protein sequence. The sequence includes mutations G19F, H25N, F26Y, and Y120F.

SEQ ID NO: 54 provides an exemplary mutant FGF2 protein sequence. The sequence includes mutations G19F, H25N, F26Y, and T121A.

SEQ ID NO: 55 provides an exemplary mutant FGF2 protein sequence. The sequence includes mutations G19F, H25N, F26Y, R116A, S117A, R118A, K119A, Y120F, and T121A.

SEQ ID NO: 56 provides an exemplary mutant FGF2 protein sequence. The sequence includes mutations G19F, H25N, F26Y, R116A, S117A, R118A, K119E, Y120F, and T121A.

SEQ ID NO: 57 provides an exemplary mutant FGF2 protein sequence. The sequence includes mutations G19F, H25N, F26Y, and R53A.

SEQ ID NO: 58 provides an exemplary mutant FGF2 protein sequence. The sequence includes mutations G19F, H25N, F26Y, and Y120A.

SEQ ID NO: 59 provides an exemplary mutant FGF2 protein sequence. The sequence includes mutations G19F, H25N, F26Y, and W123A.

SEQ ID NO: 60 provides an exemplary mutant FGF2 protein sequence. The sequence includes mutations G19F, H25N, F26Y, and R118T.

SEQ ID NO: 61 provides an exemplary mutant FGF2 protein sequence. The sequence includes mutations G19F, H25N, F26Y, and S122A.

SEQ ID NO: 62 provides an exemplary mutant FGF2 protein sequence. The sequence includes mutations G19F, H25N, F26Y, R53A, R118T, Y120A, T121A, S122A and W123A.

SEQ ID NO: 63 provides an exemplary mutant FGF2 protein sequence. The sequence includes mutations G19F, H25N, F26Y, and S73Y.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® and UniProt Accession Nos. referred to herein are the sequences available at least as early as January 22, 2016, and are incorporated by reference. All references herein, including journal articles, patents and patent applications, are incorporated by reference.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration: To provide or give a subject an agent, such as a mutated FGF2 protein disclosed herein, by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes.

C-terminal portion: A region of a protein sequence that includes a contiguous stretch of amino acids that begins at or near the C-terminal residue of the protein. A C-terminal portion of the protein can be defined by a contiguous stretch of amino acids (e.g., a number of amino acid residues).

Diabetes mellitus: A group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes”. Type 2 diabetes results from insulin resistance, a condition in which cells fail to use insulin properly, sometimes combined with an absolute insulin deficiency. This form is also called “non insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and in some examples diagnosed by demonstrating any one of:
a. Fasting plasma glucose level≥7.0 mmol/l (126 mg/dl);
b. Plasma glucose≥11.1 mmol/l (200 mg/dL) two hours after a 75 g oral glucose load as in a glucose tolerance test;
c. Symptoms of hyperglycemia and casual plasma glucose≥11.1 mmol/l (200 mg/dl);
d. Glycated hemoglobin (Hb A1C)≥6.5%

Effective amount or Therapeutically effective amount: The amount of agent, such as a mutated FGF2 protein (or nucleic acid encoding such) disclosed herein, that is an amount sufficient to prevent, treat (including prophylaxis), reduce and/or ameliorate the symptoms and/or underlying causes of any of a disorder or disease. In one embodiment, an “effective amount” is sufficient to reduce or eliminate a symptom of a disease, such as one or more metabolic disorders, such as diabetes (such as type II diabetes), for example by lowering blood glucose.

Fibroblast Growth Factor 2 (FGF2): e.g., OMIM 134920. Includes FGF2 nucleic acid molecules and proteins. FGF2 is a paracrine FGF that binds to the FGF receptor, and is also known as the basic FGF. FGF2 is present in basement membranes and in the subendothelial extracellular matrix of blood vessels. It stays bound to the extracellular matrix in the absence of a stress signal. FGF2 sequences are publically available, for example from GenBank® sequence database (e.g., GenBank® Accession Nos. NM_002006.4, NM_019305.2, NM_174056.3, and NM_008006.2 provide exemplary FGF2 nucleic acid sequences and GenBank® Accession Nos. NP_001997.5, NP_062178.1, NP_776481.1, and NP_032032.1 provide exemplary FGF2 protein sequences). One of ordinary skill in the art can identify additional FGF2 nucleic acid and protein sequences, including FGF2 variants.

Specific examples of native FGF2 sequences are provided in SEQ ID NOS: 1-5. A native FGF2 sequence is one that does not include a mutation that alters the normal activity of the protein (e.g., activity of SEQ ID NO: 3 or amino acids 10-154 of SEQ ID NO: 5). One of ordinary skill in the art can identify additional native FGF2 nucleic acid and protein sequences. A mutated FGF2 is a variant of FGF2 with different or altered biological activity, such as reduced mitogenicity (e.g., a variant of any of SEQ ID NOS: 1-5, such as one having at least 90%, at least 95%, at least 96%, at least 97%, at least 98% or at least 99% sequence identity to any of SEQ ID NOS: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63 and has reduced mitogenicity relative to a native FGF2). In one example, such a variant includes an N-terminal truncation, at least one point mutation, or combinations thereof, such as changes that decrease mitogenicity of FGF2, lower blood glucose, or combinations thereof. Specific exemplary FGF2 mutant proteins are shown in SEQ ID NOS: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62 and 63.

Fibroblast Growth Factor 19 (FGF19): e.g., OMIM 603891. Includes FGF19 nucleic acid molecules and proteins (known as FGF15 in rodents). FGF19 is a hormone-like protein that regulates carbohydrate, lipid and bile acid metabolism. FGF19 acts through receptor complex FGFR4-β-Klotho (KLB) to regulate bile acid metabolism. The murine ortholog of FGF19 is Fgf15. FGF19 sequences are publically available, for example from the GenBank® sequence database (e.g., Accession Nos. NP_005108.1, AAQ88669.1, NP_032029.1, NP_570109.1, and NP_032029.1 provide exemplary native FGF19 protein sequences, while Accession Nos. AY358302.1, NM_008003.2, and NM_005117.2 provide exemplary native FGF19 nucleic acid sequences). A specific example is provided in SEQ ID NO: 45. One of ordinary skill in the art can identify additional native FGF19 nucleic acid and protein sequences. M70 is a modified mature FGF19 with 3 amino acid substitutions (A305, G31S, and H33L) and a 5-amino acid deletion as shown below (FGF19 fragment is aa 23-42 of SEQ ID NO: 45; M70 fragment is SEQ ID NO: 47):

| | |
|---|---|
| FGF19 | RPLAFSDAGPHVHYGWGDPI- |
| M70 | MR-----DSSPLVHYGWGDPI- |

Fibroblast Growth Factor 21 (FGF21): e.g., OMIM 609436. Includes FGF21 nucleic acid molecules and proteins. FGF21 stimulates glucose updated in adipocytes. FGF21 sequences are publically available, for example from the GenBank® sequence database (e.g., Accession Nos. AAQ89444.1, NP_061986, and AAH49592.1 provide exemplary native FGF21 protein sequences, while Accession Nos. AY359086.1 and BC049592 provide exemplary native FGF21 nucleic acid sequences). One of ordinary skill in the art can identify additional FGF21 nucleic acid and protein sequences, including FGF21 variants. An exemplary FGF21 protein sequence is shown in SEQ ID NO: 46.

Host cells: Cells in which an exogous DNA expressed, for example from a vector. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. Thus, host cells can be transgenic, in that they include nucleic acid molecules that have been introduced into the cell, such as a nucleic acid molecule encoding a mutant FGF2 protein disclosed herein.

Isolated: An “isolated” biological component (such as a mutated FGF2 protein or nucleic acid molecule) has been substantially separated, produced apart from, or purified away from other biological components in the cell of the organism in which the component occurs, such as other chromosomal and extrachromosomal DNA and RNA, and proteins. Nucleic acids molecules and proteins which have been “isolated” thus include nucleic acids and proteins purified by standard purification methods. The term also embraces nucleic acid molecules and proteins prepared by recombinant expression in a host cell as well as chemically synthesized nucleic acids and proteins. A purified or isolated cell, protein, or nucleic acid molecule can be at least 70%, at least 80%, at least 90%, at least 95%, at least 96%, at least 97%, at least 98%, or at least 99% pure.

Mammal: This term includes both human and non-human mammals. Similarly, the term “subject” includes both human and veterinary subjects (such as cats, dogs, cows, and pigs).

Metabolic disorder/disease: A disease or disorder that results from the disruption of the normal mammalian process of metabolism. Includes metabolic syndrome.

Examples include but are not limited to: (1) glucose utilization disorders and the sequelae associated therewith, including diabetes mellitus (Type I and Type-2), gestational diabetes, hyperglycemia, insulin resistance, abnormal glucose metabolism, “pre-diabetes” (Impaired Fasting Glucose (IFG) or Impaired Glucose Tolerance (IGT)), and other physiological disorders associated with, or that result from, the hyperglycemic condition, including, for example, histopathological changes such as pancreatic β-cell destruction; (2) dyslipidemias and their sequelae such as, for example, atherosclerosis, coronary artery disease, cerebrovascular disorders and the like; (3) other conditions which may be associated with the metabolic syndrome, such as obesity and elevated body mass (including the co-morbid conditions thereof such as, but not limited to, nonalcoholic fatty liver disease (NAFLD), nonalcoholic steatohepatitis (NASH), and polycystic ovarian syndrome (PCOS)), and also include thromboses, hypercoagulable and prothrombotic states (arterial and venous), hypertension, cardiovascular disease, stroke and heart failure; (4) disorders or conditions in which inflammatory reactions are involved, including atherosclerosis, chronic inflammatory bowel diseases (e.g., Crohn's disease and ulcerative colitis), asthma, lupus erythematosus, arthritis, or other inflammatory rheumatic disorders; (5) disorders of cell cycle or cell differentiation processes such as adipose cell tumors, lipomatous carcinomas including, for example, liposarcomas, solid tumors, and neoplasms; (6) neurodegenerative diseases and/or demyelinating disorders of the central and peripheral nervous systems and/or neurological diseases involving neuroinfiammatory processes and/or other peripheral neuropathies, including Alzheimer's disease, multiple sclerosis, Parkinson's disease, progressive multifocal leukoencephalopathy and Guillian-Barre syndrome; (7) skin and dermatological disorders and/or disorders of wound healing processes, including erythemato-squamous dermatoses; and (8) other disorders such as syndrome X, osteoarthritis, and acute respiratory distress syndrome. Other examples are provided in WO 2014/085365 (herein incorporated by reference).

In specific examples, the metabolic disease includes one or more of (such as at least 2 or at least 3 of): diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension).

N-terminal portion: A region of a protein sequence that includes a contiguous stretch of amino acids that begins at or near the N-terminal residue of the protein. An N-terminal portion of the protein can be defined by a contiguous stretch of amino acids (e.g., a number of amino acid residues).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a mutated FGF2 coding sequence). Generally, operably linked DNA sequences are contiguous and, where necessary to join two protein coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the disclosed mutated FGF2 proteins (or nucleic acid molecules encoding such) herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: Ann array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant: A recombinant nucleic acid molecule is one that has a sequence that is not naturally occurring (e.g., a mutated FGF2) or has a sequence that is made by an artificial combination of two otherwise separated segments of sequence. This artificial combination can be accomplished by routine methods, such as chemical synthesis or by the artificial manipulation of isolated segments of nucleic acids, such as by genetic engineering techniques. Similarly, a recombinant protein is one that has a sequence that is not naturally occurring, such as one encoded for by a recombinant nucleic acid molecule. Similarly, a recombinant or transgenic cell is one that contains a recombinant nucleic acid molecule and expresses a recombinant protein.

Sequence identity of amino acid sequences: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are. Homologs of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Variants of the mutated FGF2 proteins and coding sequences disclosed herein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, a mutant FGF2 protein disclosed herein can have at least 80%, at least 85%, at least 90%, at least 91%, at least 92%, at least 93%, at least 94%, at least 95%, at least 96%, at least 97%, at least 98% or at least 99% sequence identity to any of SEQ ID NOS: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, and 63, and retain the ability to reduce blood glucose levels in vivo (but is not a native FGF2 sequence, such as SEQ ID NO: 2, 3 or 5).

Subject: Any mammal, such as humans, non-human primates, pigs, sheep, cows, dogs, cats, rodents and the like which is to be the recipient of the particular treatment, such as treatment with a mutated FGF2 protein (or corresponding nucleic acid molecule) provided herein. In two non-limiting examples, a subject is a human subject or a murine subject. In some examples, the subject has one or more metabolic diseases, such as diabetes (e.g., type 2 diabetes, non-type 2 diabetes, type 1 diabetes, latent autoimmune diabetes (LAD), maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular disease (e.g., hypertension), or combinations thereof. In some examples, the subject has elevated blood glucose.

Transduced and Transformed: A virus or vector “transduces” a cell when it transfers nucleic acid into the cell. A cell is “transformed” or “transfected” by a nucleic acid introduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses {Wolff, J. A., ed, Gene Therapeutics, Birkhauser, Boston, USA (1994)}. In the case of infection by retroviruses, the infecting retrovirus particles are absorbed by the target cells, resulting in reverse transcription of the retroviral RNA genome and integration of the resulting provirus into the cellular DNA.

Transgene: An exogenous gene, for example supplied by a vector. In one example, a transgene includes a mutated FGF2 coding sequence.

Vector: A nucleic acid molecule as introduced into a host cell, thereby producing a transformed host cell. A vector may include nucleic acid sequences that permit it to replicate in the host cell, such as an origin of replication. A vector may also include one or more mutated FGF2 coding sequences and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### Overview

It is shown herein that mutants of fibroblast growth factor (FGF)2 can be used to reduce blood glucose in a mammal. Based on these observations, methods for reducing blood glucose in a mammal, for example to treat a metabolic disease, are disclosed. Such FGF2 mutants can have an N-terminal truncation, point mutation(s), or combinations thereof, for example to gain the ability to reduce blood glucose in a mammal, to reduce the mitogenic activity of the native FGF2 protein, or combinations thereof. These mutant FGF2 proteins (or nucleic acids encoding such proteins) can be used to reduce blood glucose in a mammal, for example to treat a metabolic disease. Such FGF2 mutants can be used alone or in combination with other agents, such as other glucose reducing agents, such as thiazolidinedione.

The disclosed FGF2 mutants can have an N-terminal truncation (wherein in some examples one or more deleted residues can be replaced with other residues, such as corresponding residues from FGF21 or from the engineered analog of FGF19 termed M70), one or more point mutations, or combinations thereof, for example to reduce the mitogenic activity and bone toxicity of the native FGF2 protein. Such FGF2 mutants can be used alone or in combination with other agents, such as other glucose reducing agents, such as thiazolidinedione. In some examples, the disclosed methods can be used in a mammal with the result to reduce in triglycerides, decrease in insulin resistance, reduce hyperinsulinemia, increase glucose tolerance, reduce hyperglycemia, or combinations thereof.

Thus, methods of reducing blood glucose, treating one or more metabolic diseases, or combinations thereof, in a mammal are provided. In some examples such methods include administering a therapeutically effective amount of a mutated mature FGF2 protein to the mammal, or a nucleic acid molecule encoding the mutated mature FGF2 protein or a vector comprising the nucleic acid molecule, thereby reducing the blood glucose, treating the one or more metabolic diseases, or combinations thereof. Exemplary metabolic diseases that can be treated with the disclosed methods include but are not limited to: type 2 diabetes, non-type 2 diabetes, type 1 diabetes, polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular diseases (e.g., hypertension), latent autoimmune diabetes (LAD), or maturity onset diabetes of the young (MODY).

Also provided are methods of reducing fed and fasting blood glucose, improving insulin sensitivity and glucose tolerance, reducing systemic chronic inflammation, ameliorating hepatic steatosis, or combinations thereof, in a mammal. Such methods can include administering a therapeutically effective amount of a mutated mature FGF2 protein to the mammal, or a nucleic acid molecule encoding the mutated FGF2 protein or a vector comprising the nucleic acid molecule, thereby reducing fed and fasting blood glucose, improving insulin sensitivity and glucose tolerance, reducing systemic chronic inflammation, ameliorating hepatic steatosis, reduce one or more non-HDL lipid levels, or combinations thereof, in a mammal. In some examples, the fed and fasting blood glucose is reduced in the treated subject by at least 5%, at least 10%, at least 20%, at least 30%, at least 50%, or at least 75%, as compared to an absence of administration of mutant FGF2. In some examples, insulin sensitivity and glucose tolerance is increased in the treated subject by at least 10%, at least 20%, at least 30%, at least 50%, at least 75%, or at least 90% as compared to an absence of administration of mutant FGF2. In some examples, systemic chronic inflammation is reduced in the treated subject by at least 10%, at least 20%, at least 30%, at least 50%, at least 75%, or at least 90% as compared to an absence of administration of mutant FGF2. In some examples, hepatic steatosis is reduced in the treated subject by at least 10%, at least 20%, at least 30%, at least 50%, at least 75%, or at least 90% as compared to an absence of administration of mutant FGF2. In some examples, one or more lipids (such as a non-HDL, for example IDL, LDL and/or VLDL) are reduced in the treated subject by at least 10%, at least 20%, at least 30%, at least 50%, at least 75%, or at least 90% as compared to an absence of administration of mutant FGF2. In some examples, triglyceride and/or cholesterol levels are reduced with the mutated FGF2 by at least 10%, at least 20%, at least 30%, at least 50%, at least 75%, or at least 90% as compared to native FGF2. In some examples, combinations of these reductions are achieved.

The mutated mature FGF2 protein used in the disclosed methods can include a deletion of at least six contiguous N-terminal amino acids, at least one point mutation, or combinations thereof. Specific examples of such proteins are provided herein. In some examples, the mutated mature FGF2 protein has reduced mitogenic activity compared to native FGF2 (e.g., SEQ ID NO: 3 or amino acids 10-154 of SEQ ID NO: 5), has greater glucose lowering activity compared to native FGF2, or combinations thereof. In some examples, mitogenic activity, is reduced with the mutated FGF2 by at least 10%, at least 20%, at least 30%, at least 50%, at least 75%, at least 90%, or at least 95%, as compared to native FGF2. In some examples, glucose lowering activity is increased with the mutated FGF2 by at least 10%, at least 20%, at least 30%, at least 50%, at least 75%, or at least 90% as compared to native FGF2.

In some examples, the mutated mature FGF2 protein used in the disclosed methods has at least 6, at least 7, at least 8, at least 9, at least 10, at least 11, at least 12, at least 13, at least 14, at least 15, at least 16, at least 17, at least 18, at least 10, at least 15, at least 20, or at least 27 contiguous N-terminal amino acids deleted from the mature native FGF2 protein, wherein the mutated FGF2 protein has reduced mitogenic activity as compared to native mature FGF2 protein. In some examples, the deleted N-terminal amino acids are replaced with other amino acids, such as corresponding amino acids from an engineered analog of FGF19 (M70) or FGF21. In some examples, deleted N-terminal amino acids are replaced with at least 2, at least 3, at least 4, at least 5, at least 6, at least 7, at least 8, at least 9, or at least 10 (such as 1, 2, 3, 4, 5, 6, 7, 8, 9, 10, 11, 12, 13, 14, 15, 16, 17, 18, 19 or 20) other residues, such as those from an engineered analog of FGF19 (M70) or FGF21. In some examples, the mutated mature FGF2 protein used in the disclosed methods has at least one point mutation at one or more of G19, H25, and F26, wherein the numbering refers to the sequence shown SEQ ID NO: 3. Exemplary point mutations are provided in Table 1. In a specific example, at least one point mutation includes a mutation at G19, H25, F26, K30, Y33, R53, Q65, S73, C96, E105, N111, Y112, N113, R116, S117, R118, K119, Y120, T121, S122, W123, K128, R129, Q132, K134, and/or S137 (such as an FGF2 mutant that includes G19F, H25N, and F26Y), wherein the numbering refers to the sequence shown SEQ ID NO: 3, and wherein the mutated FGF2 protein has decreased mitogenicity as compared to wild-type mature FGF2 protein. In some examples, the mutated mature FGF2 protein used in the disclosed methods has a combination of N-terminal deletions and amino acid substitutions. Specific exemplary mutated mature FGF2 proteins include those having at least 80%, at least 85%, at least 90%, at least 92%, at least 95%, at least 96%, at least 97%, at least 98%, or at least 99% sequence identity to any of SEQ ID NOs: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63, and which have reduced mitogenic activity, increased ability to reduce blood glucose in vivo, or combinations thereof. In a specific example, the mutated mature FGF2 protein includes or consists of SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63.

Any routine method of administration can be used, such as subcutaneous, intraperitoneal, intramuscular, or intravenous. In some examples, the therapeutically effective amount of the mutated mature FGF2 protein is at least 0.1 mg/kg (such as at least 0.2 mg/kg, 0.5 mg/kg, at least 1 mg/kg, at least 2 mg/kg, at least 5 mg/kg or at least 10 mg/kg, such as 0.1 mg/kg to 100 mg/kg, 0.1 mg/kg to 0.5 mg/kg, 0.1 mg/kg to 1 mg/kg, 1 mg/kg to 5 mg/kg, 1 mg/kg to 10 mg/kg or 2 mg/kg to 50 mg/kg).

Exemplary subjects that can be treated with the disclosed methods include mammals, such as human and veterinary subjects, such as a cat or dog or livestock. In some examples, the mammal, such as a human, cat or dog, has diabetes. In some examples, the mammal, such as a human, cat or dog, has one or more metabolic diseases.

Provided herein are mutated FGF2 proteins that can include an N-terminal deletion, one or more point mutations (such as amino acid substitutions, deletions, additions, or combinations thereof), or combinations of N-terminal deletions and point mutations. In a specific example, an isolated mutated mature FGF2 protein has at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63 (but is not a native sequence and thereby acquires the ability to reduce blood glucose in vivo).

Also provided are method of using FGF2 mutant proteins (or their nucleic acid coding sequences) use to lower glucose, for example to treat a metabolic disease. In some examples, mutations in FGF2 reduce the mitogenicity of mature FGF2 (e.g., SEQ ID NO: 3), such as a reduction of at least 20%, at least 50%, at least 75% or at least 90% relative to a native mature FGF2 (e.g., SEQ ID NO: 3 or amino acids 10-154 of SEQ ID NO: 5).

In some examples, the mutant FGF2 protein is a truncated version of the mature protein (e.g., SEQ ID NO: 3 or amino acids 10-154 of SEQ ID NO: 5), which can include for example deletion of at least 5, at least 6, at least 9, at least 10, at least 11, at least 12, at least 13, at least 14, at least 15, at least 16, at least 17, at least 18, at least 19, at least 20, or at least 27 consecutive N-terminal amino acids, such as the N-terminal 5, 6, 7, 8, 9, 10, 11, 12, 13, 14, 15, 16, 17, 18, 19, 20, 21, 22, 23, 24, 25, 26, or 27 consecutive N-terminal amino acids of mature FGF2. In some examples, such an N-terminally deleted FGF2 protein has reduced mitogenic activity as compared to a native mature FGF2 protein. Examples of FGF2 proteins with N-terminal truncations are shown in SEQ ID NOS: 9, 10, 11, 24, 25, 26, 27, 28, 29, 30, 31, 32, 33, 34, 35, 36, 37, 38, 39, 40, 41, 42, 43 and 44.

In some examples, mutations in FGF2 increase the thermostability of mature or truncated FGF2, such as an increase of at least 20%, at least 50%, at least 75% or at least 90%. Exemplary mutations that can be used to increase the thermostability of mutated FGF2 include but are not limited to one or more of: Q65L/I/V, C96S/T, N111A/G, T121/S, K128N, and S137P, wherein the numbering refers to SEQ ID NO: 3. For example, mutated FGF2 can be mutated to increase the thermostability of the protein compared to an FGF2 protein without the modification. Methods of measuring thermostability are known in the art.

In some examples, the mutant FGF2 protein is a mutated version of the mature protein (e.g., SEQ ID NO: 3 or amino acids 10-154 of SEQ ID NO: 5), such as one containing at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 amino acid substitutions, such as 1-20, 1-10, 2-4, 4-8, 5-25, 1, 2, 3, 4, 5, 6, 7, 8, 9, 10, 11, 12, 13, 14, 15, 16, 17, 18, 19, 20, 21, 22, 23, 24, or 25 amino acid substitutions. Examples of FGF2 proteins with point mutations are shown in SEQ ID NOS: 8, 12, 13, 14, 15, 16, 17, 18, 19, 20, 21, 22, 23, 48, 49, 50, 51, 52, 53, 54, 55, 56, 57, 58, 59, 60, 61, 62, and 63. In some examples, the mutant FGF2 protein includes deletion of one or more amino acids, such as deletion of 1-10, 10-20, 4-8, 5-10, 1, 2, 3, 4, 5, 6, 7, 8, 9, 10, 11, 12, 13, 14, 15, 16, 17, 18, 19, 20, 21, 22, 23, 24, 25, 26, or 27 amino acids. In some examples, one or more (such as 2-27, 2-10, 4-9, or 4-7) of the deleted N-terminal residues are replaced with other residues, such as those from an engineered analog of FGF19 (M70) or FGF21 (such as 2-27, 3-10, or 2-7, for example 4, 5, 6, 7, 8, 9, or 10 contiguous amino acids from an engineered analog of FGF19 (M70) or FGF21). In some examples, the mutant FGF2 protein includes a combination of amino acid substitutions and deletions, such as at least 1 substitution and at least 1 deletion, such as 1 to 10 substitutions with 1 to 20 deletions. Examples of such combinations are shown in SEQ ID NOS: 9, 10, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, and 63.

Exemplary FGF2 mutations are shown in Table 1 below, with amino acids referenced to SEQ ID NO: 3 (155 aa form). On skilled in the art will recognize the corresponding mutations can be made to other FGF2 proteins, such as those from other species (e.g., H25 of SEQ ID NO: 3 corresponds to aa H24 of SEQ ID NO: 5). One skilled in the art will recognize that these mutations can be used singly, or in combination (such as 1-18, 1-10, 3-10, 1-2, 2-4, 1-5, 1, 2, 3, 4, 5, 6, 7, 8, 9, 10, 11, 12, 13, 14, 15, 16, 17,18, 19, 20, 21, 22, 23, 24, 25, or 26 of these amino acid substitutions). In addition, these substitutions can be combined with N-terminal truncations/replacements.

TABLE 1
Exemplary FGF2 mutations

| Location of Position in FGF2 (SEQ ID NO: 3) | Mutation Citation |
|---|---|
| G19 | G19F |
| H25 | H25N |
| F26 | F26Y |
| K30 | K30V |
| Y33 | Y33V |
| R53 | R53E, R53A |
| Q65 | Q65L/I/V/A |
| S73 | S73Y |
| C96 | C96S/T/A |
| E105 | E105V |
| N111 | N111A/G |
| Y112 | Y112V |
| N113 | N113V |
| R116 | R116A |
| S117 | S117A |
| R118 | R118A, R118T |
| K119 | K119A, K119E |
| Y120 | Y120F, Y120A |
| T121 | T121S, T121A |
| S122 | S122A |
| W123 | W123A |
| K128 | K128N/D |
| R129 | R129Q |
| Q132 | Q132R |
| K134 | K134V |
| S137 | S137P |

In some examples, the mutant FGF2 protein includes mutations at one or more of the following positions, such as 1, 2, 3, 4, 5, 6, 7, 8, 9, 10, 11, 12, 13, 14, 15, 16, 17, 18, 19, 20, 21, 22, 23, 24, 25, or 26 of these positions: G19, H25, F26, K30, Y33, R53, Q65, S73, C96, E105, N111, Y112, N113, R116, S117, R118, K119, Y120, T121,

S122, W123, K128, R129, Q132, K134, and S137 (wherein the numbering refers to SEQ ID NO: 3), such as one or more of G19F, H25N, F26Y, K3OV, Y33V, R53E, R53A, Q65L, Q651, Q65V, Q65A, S73Y, C96S, C96T, C96A, E105V, N111A, N111G, Y112V, N113V, R116A, S117A, R118A, R118T, K119A, K119E, Y120F, Y120A, T121A, T121S, S122A, W123A, K128N K128D, R129Q, Q132R, R134V, and S137P (such as 1, 2, 3, 4, 5, 6, 7, 8, 9, 10, 11, 12, 13, 14, 15, 16, 17, 18, 19, 20, 21, 22, 23, 24, 25, or 26 of these mutations). One skilled in the art will appreciate that such as mutant FGF2 protein can include other changes, such as 1-20, 1-10, or 1-5 conservative amino acid substitutions that do not adversely affect the function of the mutated protein (such as 1, 2, 3, 4, 5, 6, 7, 8, 9, or 10 conservative amino acid substitutions), and/or deletion of up to and including 27 N-terminal amino acids (which can be replaced with one or more other amino acids), such that the ability of the mutant FGF2 protein to reduce blood glucose in vivo is retained.

In one example, an FGF2 mutant protein includes an K128N mutation to improve thermal stability. In one example, an FGF2 mutant protein includes an K128D mutation (for example alone or in combination with R129Q and K134V) to reduce heparan sulfate binding affinity.

In one example, an FGF2 mutant protein includes one or more mutations to reduce mitogenicity, such as a mutation at R53, R116, S117, R118, K119, Y120, T121, S122 or W123 (or combinations thereof, such as 1, 2, 3, 4, 5, 6, 7, 8 or 9 of these). Specific exemplary mutations that can be made at these positions are shown in Table 1. In one example, an FGF2 mutant protein includes SEQ ID NO: 8 with one or more of these additional mutations (such as 1, 2, 3, 4, 5, 6, 7, 8 or 9 of these mutations).

In some examples, the mutant FGF2 protein includes at least 20, at least 30, at least 40, or at least 50, consecutive amino acids of mature FGF2 (e.g., of SEQ ID NO: 3 or amino acids 10-154 of SEQ ID NO: 5), such as in the region of amino acids 28 to 155 of SEQ ID NO: 3 (the minimal folded domain) (which in some examples can include deletion of 1 to 27 N-terminal amino acids in combination with 1-5, 1-10, 2-8 or 1-20 point mutations, such as substitutions, deletions, or additions).

In some examples, the mutant FGF2 protein includes both an N-terminal truncation and point mutations, such as deletion of at least four N-terminal amino acids (such as deletion of 4, 5, 6, 7, 8, 9, 10, 11, 12 ,13, 14, 15, 16, 17, 18, 19, 20, 21, 22, 23, 24, 25, 26, or 27 contiguous N-terminal amino acids) and at least one point mutation (such as at least 2, at least 4, at least 5, at least 8, at least 10, at least 15, at least 20, or at least 30 point mutations, for example 1, 2, 3, 4, 5, 6, 7, 8, 9, 10, 11, 12 ,13, 14, 15, 16, 17, 18, 19, 20, 21, 22, 23, 24, 25 or 26 point mutations). Specific exemplary FGF2 mutant proteins are shown in SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62 and 63. In some examples, the FGF2 mutant includes an N-terminal deletion, but retains a methionine at the N-terminal position. In some examples, the FGF2 mutant is 120-200 or 140-160 amino acids in length.

In some examples, the FGF2 mutant protein includes at least 80% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. Thus, the FGF2 mutant protein can have at least 90%, at least 95%, at least 96%, at least 97%, at least 98% or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. In some examples, the FGF2 mutant protein includes or consists of SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. The disclosure encompasses variants of the disclosed FGF2 mutant proteins, such as SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63 having 1 to 8, 2 to 10, 1 to 5, 1 to 6, or 5 to 10 mutations, such as one or more of those in Table 1, for example in combination with conservative amino acid substitutions.

Also provided are isolated nucleic acid molecules encoding the disclosed mutated FGF2 proteins, such as a nucleic acid molecule encoding a protein having at least 80%, at least 85%, at least 90%, at least 95%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. Based on the coding sequence of native FGF2 shown in SEQ ID NOS: 1 and 4, one skilled in the art can generate a coding sequence of any FGF2 mutant provided herein. Vectors and cells that include such nucleic acid molecules are also provided. For example, such nucleic acid molecules can be expressed in a host cell, such as a bacterium or yeast cell (e.g., E. coli), thereby permitting expression of the mutated FGF2 protein. The resulting mutated FGF2 protein can be purified from the cell.

Methods of using the disclosed mutated FGF2 proteins (or nucleic acid molecules encoding such), are provided. As discussed herein, the mutated mature FGF2 protein can include a deletion of at least six contiguous N-terminal amino acids, at least one point mutation, or combinations thereof. For example, such methods include administering a therapeutically effective amount of a disclosed mutated FGF2 protein (such as at least 0.01, at least 0.1 mg/kg, or at least 0.5 mg/kg) (or nucleic acid molecules encoding such) to reduce blood glucose in a mammal, such as a decrease of at least 5%, at least 10%, at least 25% or at least 50%.

In some examples, use of the FGF2 mutants disclosed herein does not lead to (or significantly reduces, such as a reduction of at least 20%, at least 50%, at least 75%, or at least 90%) the adverse side effects observed with thiazolidinediones (TZDs) therapeutic insulin sensitizers, including weight gain, increased liver steatosis and bone fractures (e.g., reduced affects on bone mineral density, trabecular bone architecture and cortical bone thickness).

### Mutated FGF2 Proteins

The present disclosure provides mutated FGF2 proteins that can include an N-terminal deletion, one or more point mutations (such as amino acid substitutions, deletions, additions, or combinations thereof), or combinations of N-terminal deletions and point mutations. Such proteins and corresponding coding sequences can be used in the methods provided herein. In some examples, the disclosed FGF2 mutant proteins have reduced mitogenicity compared to mature native FGF2 (e.g., SEQ ID NO: 3 or amino acids 10-154 of SEQ ID NO: 5), such as a reduction of at least 20%, at least 50%, at least 75% or at least 90%. Methods of measuring mitogenicity are known in the art.

In some examples, the mutant FGF2 protein is a truncated version of the native mature protein (e.g., SEQ ID NO: 3 or amino acids 10-154 of SEQ ID NO: 5), which can include for example deletion of at least 5, at least 6, at least 7, at least 8, at least 9, at least 10, at least 11, at least 12, at least 13, at least 14, at least 15, at least 16, at least 17, at least 18, at least 19, at least 20 or at least 27 consecutive N-terminal amino acids. Thus, in some examples, the mutant FGF2 protein is a truncated version of the mature protein (e.g., SEQ ID NO: 3 or amino acids 10-154 of SEQ ID NO: 5), such a deletion of the N-terminal 5, 6, 7, 8, 9, 10, 11, 12, 13, 14, 15, 16, 17, 18, 19, 20, 21, 22, 23, 24, 25, 26, or 27 amino acids shown in SEQ ID NO: 3 or amino acids 10-154 of SEQ ID NO: 5. In some examples one or more of the amino acids deleted from the N-terminal end of FGF2 are replaced with other amino acids, such as at least 4, at least 5, or at least 10 corresponding contiguous amino acids from an engineered analog of FGF19 (M70) or FGF21. Examples of N-terminally truncated FGF2 proteins are shown in SEQ ID NOS: 9-11 and 24-44. In some examples, the FGF2 mutant includes an N-terminal deletion, but retains a methionine at the N-terminal position. In some examples, such an N-terminally deleted FGF2 protein has reduced mitogenic activity as compared to wild-type mature FGF2 protein.

Thus, in some examples, the mutant FGF2 protein includes at least 30, at least 40, or at least 50 consecutive amino acids of mature FGF2 (e.g., of SEQ ID NO: 3 or amino acids 10-154 of SEQ ID NO: 5), such as in the region of amino acids 46 to 95 of mature FGF2 (e.g., of SEQ ID NO: 3), (which in some examples can include 1-5, 1-10 or 1-20 point mutations, such as substitutions, deletions, or additions).

In some examples, the mutant FGF2 protein is a mutated version of the mature protein (e.g., SEQ ID NO: 3 or amino acids 10-154 of SEQ ID NO: 5), or a N-terminal truncation of the mature protein, such as one containing at least 1, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acid substitutions, such as 1-20, 1-10, 4-8, 5-12, 5-10, 5-25, 1, 2, 3, 4, 5, 6, 7, 8, 9, 10, 11, 12, 13, 14, or 15 amino acid substitutions. For example, point mutations can be introduced into an FGF2 sequence to decrease mitogenicity and/or increase stability, compared to the FGF2 protein without the modification. Specific exemplary point mutations that can be used are shown above in Table 1.

In some examples, the mutant FGF2 protein includes mutations (such as a substitution or deletion) at one or more of the following positions, such as 1, 2, 3, 4, 5, 6, 7, 8, 9, 10, 11, 12, 13, 14, 15, 16, 17, 18, 19, 20, 21, 22, 23, 24, 25, or 26 of these positions: G19, H25, F26, K30, Y33, R53, Q65, S73, C96, E105, N111, Y112, N113, R116, S117, R118, K119, Y120, T121, S122, W123, K128, R129, Q132, K134, and S137 (wherein the numbering refers to SEQ ID NO: 3), such as one or more of G19F, H25N, F26Y, K3OV, Y33V, R53E, R53A, Q65L, Q65I, Q65V, Q65A, S73Y C96S, C96T, C96A, E105V, N111A, N111G, Y112V, N113V, R116A, S117A, R118A, R118T, K119A, K119E, Y120F, Y120A, T121A, T121S, S122A, W123A, K128N K128D, R129Q, Q132R, R134V, and S137P (such as 1, 2, 3, 4, 5, 6, 7, 9, 10, 11, 12, 13, 14, 15, 16, 17, 18, 29, 20, 21, 22, 23, 24, 25 or 26 of these mutations). In some examples, such an FGF2 protein with one or more point mutations has reduced mitogenic activity as compared to wild-type mature FGF2 protein. Examples of FGF2 mutant proteins containing point mutations include but are not limited to the protein sequence shown in any of SEQ ID NOs: 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63.

In some examples, mutations in FGF2 increase the thermostability of mature or truncated FGF2. For example, mutations can be made at one or more of the following positions. Exemplary mutations that can be used to increase the thermostability of mutated FGF2 include but are not limited to one or more of: Q65L/I/V, C96S/T, N111A/G, T121/S, K128N, and S137P, wherein the numbering refers to SEQ ID NO: 3.

In some examples, the mutant FGF2 protein includes both an N-terminal truncation and point mutations. Specific exemplary FGF2 mutant proteins having both one or more point mutations and an N-terminal deletion are shown in SEQ ID NOs: 9, 10, 24, 25, 26, 27, 28, 29, 30, 31, 32, 33, 34, 35, 36, 37, 38, 39, 40, 41, 42, 43 and 44. In some examples, the FGF2 mutant protein includes at least 80% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. Thus, the FGF2 mutant protein can have at least 90%, at least 95%, at least 96%, at least 97%, at least 98% or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. In some examples, the FGF2 mutant protein includes or consists of SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. The disclosure encompasses variants of the disclosed FGF2 mutant proteins, such as SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63, having 1 to 20, 1 to 15, 1 to 10, 1 to 8, 2 to 10, 1 to 5, 1 to 6, 2 to 12, 3 to 12, 5 to 12, or 5 to 10 mutations, such as conservative amino acid substitutions.

In some examples, the mutant FGF2 protein has at its N-terminus a methionine.

In some examples, the mutant FGF2 protein is at least 110 amino acids in length, such as at least 120, at least 125, at least 130, at least 135, at least 140, at least 145, at least 150, at least 155, at least 160, or at least 165 amino acids in length, such as 110 to 200, 140 to 190, 140 to 170, 140 to 160, or 150 to 160 amino acids in length.

Exemplary N-terminally truncated FGF2 sequences and FGF2 point mutations that can be used to generate an FGF2 mutant protein are shown in Table 1 (as well as those provided in SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63). One skilled in the art will appreciate that any N-terminal truncation provided herein can be combined with any FGF2 point mutation in Table 1, to generate an FGF2 mutant protein. In addition, mutations can be made to the sequences shown in SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63, such as one or more of the mutations discussed herein (such as 1, 2, 3, 4, 5, 6, 7, 8, 9, 10, 11, 12, 13, 14, 15, 16, 17, 18, 19, or 20 amino acid substitutions, such as conservative amino acid substitutions, deletions, or additions).

Exemplary mutant FGF2 proteins are provided in SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63. One skilled in the art will recognize that minor variations can be made to these sequences, without adversely affecting the function of the protein (such as its ability to reduce blood glucose). For example, variants of the mutant FGF2 proteins include those having at least 90%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63, but retain the ability to treat one or more metabolic diseases, and/or decrease blood glucose in a mammal (such as a mammal with type II diabetes). Thus, variants of SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63 retaining at least 80%, at least 90%, at least 92%, at least 94%, at least 95%, at least 96%, at least 97%, at least 98% or at least 99% sequence identity, and retaining the ability to reduce blood glucose in vivo, are of use in the disclosed methods.

### FGF2

Mature forms of FGF2 (such as SEQ ID NO: 3 or amino acids 10-154 of SEQ ID NO: 5) can be mutated to control (e.g., reduce) the mitogenicity of the protein, provide glucose-lowering ability to the protein, or combinations thereof. Mutations can also be introduced into a wild-type mature FGF2 sequence that affects the stability and receptor binding selectivity of the protein.

Exemplary mature FGF2 proteins are shown in SEQ ID NOS: 3 (human) and 5 (mouse). In some examples, FGF2 includes SEQ ID NO: 3 or 5, but without the N-terminal methionine. Mutations can be introduced into a wild-type FGF2 (such as SEQ ID NO: 3 or 5). In some examples, multiple types of mutations disclosed herein are made to the FGF2 protein. Although mutations below are noted by a particular amino acid for example in SEQ ID NO: 3 or 5, one skilled in the art will appreciate that the corresponding amino acid can be mutated in any FGF2 sequence.

In one example, mutations are made to the N-terminal region of mature FGF2 (such as SEQ ID NO: 3), such as deletion of the first 5, 6, 7, 8, 9, 10, 11, 12, 13, 14, 15, 16, 17, 18, 19, 20, 21, 22, 23, 24, 25, 26, or 27 amino acids of SEQ ID NO: 3 or 5.

Mutations can be made to FGF2 (such as SEQ ID NO: 3) to reduce its mitogenic activity. In some examples, such mutations reduce mitogenic activity by at least 50%, at least 60%, at least 70%, at least 75%, at least 80%, at least 90%, at least 92%, at least 95%, at least 98%, at least 99%, or even complete elimination of detectable mitogenic activity. Methods of measuring mitogenic activity are known in the art, such as thymidine incorporation into DNA in serum-starved cells (e.g., NIH 3T3 cells) stimulated with the mutated FGF1, methylthiazoletetrazolium (MTT) assay (for example by stimulating serum-starved cells with mutated FGF2 for 24 hr then measuring viable cells), cell number quantification or BrdU incorporation. In some examples, the assay provided by Fu et al., World J. Gastroenterol. 10:3590-6, 2004; Klingenberg et al., J. Biol. Chem. 274:18081-6, 1999; Shen et al., Protein Expr Purif 81:119-25, 2011, or Zou et al., Chin. Med. J. 121:424-429, 2008 is used to measure mitogenic activity. In one example, the method of Zhou et al. (Cancer Res 74:3306-16, 2014) is used to measure HCC tumor growth. Examples of such mutations include, but are not limited to those at K30 and N113, such as K3OV and/or N113V (wherein the numbering refers to SEQ ID NO: 3). In some examples, a portion of contiguous N-terminal residues are removed, such as amino acids 1-10, 1-16, 1-20, or 1-27 of SEQ ID NO: 3, to reduce the mitogenicity of the mutated form of FGF2. The removed amino acids can be replaced with other amino acids, such as at least 4, at least 5, at least 6, at least 7, at least 8, at least 9 or at least 10 from an engineered analog of FGF19 (M70) or FGF21. Examples are shown in SEQ ID NOS: 9, 10, 11 and 24-44.

In one example, mutations are introduced to improve stability of FGF2. Methods of measuring FGF2 stability are known in the art, such as measuring denaturation of FGF2 or mutants by fluorescence and circular dichroism in the absence and presence of a 5-fold molar excess of heparin in the presence of 1.5 M urea or isothermal equilibrium denaturation by guanidine hydrochloride. In one example, the assay provided by Dubey et al., J. Mol. Biol. 371:256-268, 2007 is used to measure FGF2 stability. Examples of mutations that can be used to increase stability of the protein include, but are not limited to, one or more of Q65L/I/V, C96S/T, N111A/G, T121/S, K128N, and S137P (wherein the numbering refers to the sequence shown SEQ ID NO: 3).

In one example, mutations are introduced to improve the thermostability of FGF2 (e.g., see Xia et al., PLoS One. 2012;7(11):e48210 and Zakrzewska, J Biol Chem. 284:25388-25403, 2009).

In some examples, the mutant FGF2 protein is PEGylated at one or more positions, such as at N113 (for example see methods of Niu et al., J. Chromatog. 1327:66-72, 2014, herein incorporated by reference). Pegylation consists of covalently linking a polyethylene glycol group to surface residues and/or the N-terminal amino group. N105 is involved in receptor binding, thus is on the surface of the folded protein. As mutations to surface exposed residues could potentially generate immunogenic sequences, pegylation is an alternative method to abrogate a specific interaction. Pegylation is an option for any surface exposed site implicated in the receptor binding and/or proteolytic degradation. Pegylation can “cover” functional amino acids, e.g. R53, E105, Y112, and/or N113 (reference to SEQ ID NO: 3), as well as increase serum stability.

In some examples, the mutant FGF2 protein includes an immunoglobin FC domain (for example see Czajkowsky et al., EMBO Mol. Med. 4:1015-28, 2012, herein incorporated by reference). The conserved FC fragment of an antibody can be incorporated either n-terminal or c-terminal of the mutant FGF2 protein, and can enhance stability of the protein and therefore serum half-life. The FC domain can also be used as a means to purify the proteins on protein A or Protein G sepharose beads. This makes the FGF2 mutants having heparin binding mutations easier to purify.

### Variant Sequences

Variant FGF2 proteins, including variants of the sequences shown SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, and 63 can contain one or more mutations, such as a single insertion, a single deletion, a single substitution. In some examples, the mutant FGF2 protein includes 1-20 insertions, 1-20 deletions, 1-20 substitutions, or any combination thereof (e.g., single insertion together with 1-19 substitutions). In some examples, the disclosure provides a variant of any disclosed mutant FGF2 protein having 1, 2, 3, 4, 5, 6, 7, 8, 9, 10, 11, 12, 13, 14, 15, 16, 17, 18, 19 or 20 amino acid changes. In some examples, SEQ ID NO: 8 includes an additional 1-8 insertions, 1-15 deletions, 1-10 substitutions, or any combination thereof (e.g., 1-15, 1-4, or 1-5 amino acid deletions together with 1-10, 1-5 or 1-7 amino acid substitutions). In some examples, the disclosure provides a variant of SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 4, 44, 48, 49, 50, 51, 52, 53, 54, 55, 56, 57, 58, 59, 60, 61, 62, or 63, having 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid changes. In one example, such variant peptides are produced by manipulating the nucleotide sequence encoding a peptide using standard procedures such as site-directed mutagenesis or PCR. Such variants can also be chemically synthesized. Such variants retain the ability to lower blood glucose in vivo.

One type of modification or mutation includes the substitution of amino acids for amino acid residues having a similar biochemical property, that is, a conservative substitution (such as 1-4, 1-8, 1-10, or 1-20 conservative substitutions). Typically, conservative substitutions have little to no impact on the activity of a resulting peptide. For example, a conservative substitution is an amino acid substitution in SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63, that does not substantially affect the ability of the peptide to decrease blood glucose in a mammal. An alanine scan can be used to identify which amino acid residues in a mutant FGF2 protein, such as SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63, can tolerate an amino acid substitution. In one example, the blood glucose lowering activity of FGF2, or SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63, is not altered by more than 25%, for example not more than 20%, for example not more than 10%, when an alanine, or other conservative amino acid, is substituted for 1-4, 1-8, 1-10, or 1-20 native amino acids. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Be; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

More substantial changes can be made by using substitutions that are less conservative, e.g., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions (or other deletions or additions) can be assessed by analyzing the function of the mutant FGF2 protein, such as SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63, by analyzing the ability of the variant protein to decrease blood glucose in a mammal.

### Generation of Proteins

Isolation and purification of recombinantly expressed mutated FGF2 proteins can be carried out by conventional means, such as preparative chromatography and immunological separations. Once expressed, mutated FGF2 proteins can be purified according to standard procedures of the art, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes.

In addition to recombinant methods, mutated FGF2 proteins disclosed herein can also be constructed in whole or in part using standard peptide synthesis. In one example, mutated FGF2 proteins are synthesized by condensation of the amino and carboxyl termini of shorter fragments. Methods of forming peptide bonds by activation of a carboxyl terminal end (such as by the use of the coupling reagent N, N′-dicylohexylcarbodimide) are well known in the art.

### Mutated FGF2 Nucleic Acid Molecules and Vectors

Nucleic acid molecules encoding a mutated FGF2 protein are encompassed by this disclosure. Based on the genetic code, nucleic acid sequences coding for any mutated FGF2 protein, such as those having at least 90%, at least 92%, at least 95%, at least 96%, at least 97%, at least 98%, or at least 99% sequence identity to those shown in SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63 can be routinely generated. In some examples, such a sequence is optimized for expression in a host cell, such as a host cell used to express the mutant FGF2 protein.

In one example, a nucleic acid sequence coding for a mutant FGF2 protein has at least 80%, at least 90%, at least 92%, at least 95%, at least 96%, at least 97%, at least 99% or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63 can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same mutant FGF2 protein sequence.

Nucleic acid molecules include DNA, cDNA and RNA sequences which encode a mutated FGF2 peptide. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3^{rd} Edition, W.H. 5 Freeman and Co., NY).

Codon preferences and codon usage tables for a particular species can be used to engineer isolated nucleic acid molecules encoding a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) that take advantage of the codon usage preferences of that particular species. For example, the mutated FGF2 proteins disclosed herein can be designed to have codons that are preferentially used by a particular organism of interest.

A nucleic acid encoding a mutant FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). A wide variety of cloning and in vitro amplification methodologies are well known to persons skilled in the art. In addition, nucleic acids encoding sequences encoding a mutant FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be prepared by cloning techniques. Examples of appropriate cloning and sequencing techniques, and instructions sufficient to direct persons of skill through cloning are found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.

Nucleic acid sequences encoding a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences may be obtained by the ligation of shorter sequences.

In one example, a mutant FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) is prepared by inserting the cDNA which encodes the mutant FGF2 protein into a vector. The insertion can be made so that the mutant FGF2 protein is read in frame so that the mutant FGF2 protein is produced.

The mutated FGF2 protein nucleic acid coding sequence (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect, plant and mammalian organisms. Methods of expressing DNA sequences having eukaryotic or viral sequences in prokaryotes are well known in the art. Biologically functional viral and plasmid DNA vectors capable of expression and replication in a host are known in the art. The vector can encode a selectable marker, such as a thymidine kinase gene.

Nucleic acid sequences encoding a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be operatively linked to expression control sequences. An expression control sequence operatively linked to a mutated FGF2 protein coding sequence is ligated such that expression of the mutant FGF2 protein coding sequence is achieved under conditions compatible with the expression control sequences. The expression control sequences include, but are not limited to appropriate promoters, enhancers, transcription terminators, a start codon (i.e., ATG) in front of a mutated FGF2 protein-encoding gene, splicing signal for introns, maintenance of the correct reading frame of that gene to permit proper translation of mRNA, and stop codons.

In one embodiment, vectors are used for expression in yeast such as S. cerevisiae, P. pastoris, or Kluyveromyces lactis. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The nucleic acid molecules encoding a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can also be designed to express in insect cells.

A mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast cell lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared that encode a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63). Exemplary viral vectors include polyoma, SV40, adenovirus, vaccinia virus, adeno-associated virus, herpes viruses including HSV and EBV, Sindbis viruses, alphaviruses and retroviruses of avian, murine, and human origin. Baculovirus (Autographa californica multinuclear polyhedrosis virus; AcMNPV) vectors are also known in the art, and may be obtained from commercial sources. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

Viral vectors that encode a mutated truncated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43,44, 48, 49, 50, 51, 52, 53, 54, 55, 56, 57, 58, 59, 60, 61, 62, or 63) can include at least one expression control element operationally linked to the nucleic acid sequence encoding the mutated FGF2 protein. The expression control elements are inserted in the vector to control and regulate the expression of the nucleic acid sequence. Examples of expression control elements of use in these vectors includes, but is not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the mutated FGF2 protein in the host system. The expression vector can contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Basic techniques for preparing recombinant DNA viruses containing a heterologous DNA sequence encoding the mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) are known. Such techniques involve, for example, homologous recombination between the viral DNA sequences flanking the DNA sequence in a donor plasmid and homologous sequences present in the parental virus. The vector can be constructed for example by steps known in the art, such as by using a unique restriction endonuclease site that is naturally present or artificially inserted in the parental viral vector to insert the heterologous DNA.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding an mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression systems such as plasmids and vectors of use in producing mutated FGF2 proteins in cells including eukaryotic cells such as the COS, CHO, HeLa and myeloma cell lines.

### Cells Expressing Mutated FGF2 Proteins

A nucleic acid molecule encoding a mutated FGF2 protein disclosed herein, can be used to transform cells and make transformed cells. Thus, cells expressing a mutated FGF1protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) are disclosed. Cells expressing a mutated FGF2 protein disclosed herein, can be eukaryotic or prokaryotic. Examples of such cells include, but are not limited to bacteria, archea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, SF9 cells, C129 cells, 293 cells, Neurospora, and immortalized mammalian myeloid and lymphoid cell lines.

Cells expressing a mutated FGF2 protein are transformed or recombinant cells. Such cells can include at least one exogenous nucleic acid molecule that encodes a mutated FGF2 protein, for example a sequence encoding a mutant FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63). It is understood that all progeny may not be identical to the parental cell since there may be mutations that occur during replication. Methods of stable transfer, meaning that the foreign DNA is continuously maintained in the host cell, are known in the art.

Transformation of a host cell with recombinant DNA may be carried out by conventional techniques as are well known. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. Techniques for the transformation of yeast cells, such as polyethylene glycol transformation, protoplast transformation and gene guns are also known in the art.

### Pharmaceutical Compositions That Include Mutated FGF2 Molecules

Pharmaceutical compositions that include a mutated FGF2 protein (such as a protein generated using the mutations shown in Table 1 alone or in combination with an N-terminal deletion, such as a protein having at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) or a nucleic acid encoding these proteins, can be formulated with an appropriate pharmaceutically acceptable carrier, depending upon the particular mode of administration chosen.

In some embodiments, the pharmaceutical composition consists essentially of a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) (or a nucleic acid encoding such a protein) and a pharmaceutically acceptable carrier. In these embodiments, additional therapeutically effective agents are not included in the compositions.

In other embodiments, the pharmaceutical composition includes a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) (or a nucleic acid encoding such a protein) and a pharmaceutically acceptable carrier. Additional therapeutic agents, such as agents for the treatment of diabetes or other metabolic disorder, can be included. Thus, the pharmaceutical compositions can include a therapeutically effective amount of another agent. Examples of such agents include, without limitation, anti-apoptotic substances such as the Nemo-Binding Domain and compounds that induce proliferation such as cyclin dependent kinase (CDK)-6, CDK-4 and cyclin D1. Other active agents can be utilized, such as antidiabetic agents for example, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570, aleglitazar, farglitazar, muraglitazar, tesaglitazar, and TZD) and antagonists, PPAR-gamma/alpha modulators (such as KRP 297), alpha-glucosidase inhibitors (e.g., acarbose, voglibose), dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g. exendin-4) or amylin. Additional examples include immunomodulatory factors such as anti-CD3 mAb, growth factors such as HGF, VEGF, PDGF, lactogens, and PTHrP. In some examples, the pharmaceutical compositions containing a mutated FGF2 protein can further include a therapeutically effective amount of other FGFs, such as FGF19, heparin, or combinations thereof.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) is included in a controlled release formulation, for example, a microencapsulated formulation. Various types of biodegradable and biocompatible polymers, methods can be used, and methods of encapsulating a variety of synthetic compounds, proteins and nucleic acids, have been well described in the art (see, for example, U.S. Patent Publication Nos. 2007/0148074; 2007/0092575; and 2006/0246139; U.S. Pat. Nos. 4,522, 811; 5,753,234; and 7,081,489; PCT Publication No. WO/2006/052285; Benita, Microencapsulation: Methods and Industrial Applications, 2^{nd} ed., CRC Press, 2006).

In other embodiments, a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) is included in a nanodispersion system. Nanodispersion systems and methods for producing such nanodispersions are well known to one of skill in the art. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006. With regard to the administration of nucleic acids, one approach to administration of nucleic acids is direct treatment with plasmid DNA, such as with a mammalian expression plasmid. As described above, the nucleotide sequence encoding a mutated

FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be placed under the control of a promoter to increase expression of the protein.

Many types of release delivery systems are available and known. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), or polynucleotide encoding this protein, is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Use of a long-term sustained release implant may be particularly suitable for treatment of chronic conditions, such as diabetes. Long-term release, as used herein, means that the implant is constructed and arranged to deliver therapeutic levels of the active ingredient for at least 30 days, or at least 60 days. Long-term sustained release implants are well known to those of ordinary skill in the art and include some of the release systems described above. These systems have been described for use with nucleic acids (see U.S. Pat. No. 6,218,371). For use in vivo, nucleic acids and peptides are preferably relatively resistant to degradation (such as via endo- and exo-nucleases). Thus, modifications of the disclosed mutated FGF2 proteins, such as the inclusion of a C-terminal amide, can be used.

The dosage form of the pharmaceutical composition can be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The pharmaceutical compositions that include a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be formulated in unit dosage form, suitable for individual administration of precise dosages. In one non-limiting example, a unit dosage contains from about 1 mg to about 1 g of a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), such as about 10 mg to about 100 mg, about 50 mg to about 500 mg, about 100 mg to about 900 mg, about 250 mg to about 750 mg, or about 400 mg to about 600 mg. In other examples, a therapeutically effective amount of a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) is about 0.01 mg/kg to about 50 mg/kg, for example, about 0.5 mg/kg to about 25 mg/kg or about 1 mg/kg to about 10 mg/kg. In other examples, a therapeutically effective amount of a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) is about 0.1 mg/kg to about 5 mg/kg, for example at least 0.1 mg/kg, at least 0.5 mg/kg, 0.1 mg/kg to 1 mg/kg, 0.5 mg/kg to 5 mg/kg, 1 mg/kg to 5 mg/kg or 2 mg/kg to 5 mg/kg). In a particular example, a therapeutically effective amount of a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) includes about 1 mg/kg to about 10 mg/kg, such as about 2 mg/kg.

### Treatment Using Mutated FGF2

The disclosed mutated FGF2 proteins (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), or nucleic acids encoding such proteins, can be administered to a subject, for example to treat a metabolic disease, for example by reducing fed and fasting blood glucose, improving insulin sensitivity and glucose tolerance, reducing systemic chronic inflammation, ameliorating hepatic steatosis in a mammal, reducing hypertension, reducing non-HDL lipid and/or triglyceride levels, or combinations thereof. The disclosed mutated FGF2 proteins (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), or nucleic acids encoding such proteins, can be administered to a subject, for example to reduce glucose levels, increase insulin sensitivity, reduce insulin resistance, reduce glucagon, improve glucose tolerance, or glucose metabolism or homeostasis, improve pancreatic function, reduce triglyceride, cholesterol, IDL, LDL and/or VLDL levels, decrease blood pressure, decrease intimal thickening of the blood vessel, decrease body mass or weight gain, decrease hypertension, or combinations thereof.

Thus, the disclosed mutated FGF2 proteins can be administered to subjects having a fasting plasma glucose (FPG) level greater than about 100 mg/d and/or has a hemoglobin A1c (HbA1c) level above 6%.

The disclosed mutated FGF2 proteins (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), or nucleic acids encoding such proteins, can be administered to a subject, for example to treat a subject having a hyperglycemic condition (e.g., diabetes, such as insulin-dependent (type I) diabetes, type II diabetes, or gestational diabetes), insulin resistance, hyperinsulinemia, glucose intolerance or metabolic syndrome, or is obese or has an undesirable body mass.

The disclosed mutated FGF2 proteins (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), or nucleic acids encoding such proteins, can be administered to a subject, for example to treat other hyperglycemic-related disorders, including kidney damage (e.g., tubule damage or nephropathy), liver degeneration, eye damage (e.g., diabetic retinopathy or cataracts), and diabetic foot disorders; dyslipidemias and their sequelae such as, for example, atherosclerosis, coronary artery disease, cerebrovascular disorders and the like.

The compositions of this disclosure that include a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) (or nucleic acids encoding these molecules) can be administered to humans or other animals by any means, including orally, intravenously, intramuscularly, intraperitoneally, intranasally, intradermally, intrathecally, subcutaneously, via inhalation or via suppository. In one non-limiting example, the composition is administered via injection. In some examples, site-specific administration of the composition can be used, for example by administering a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) (or a nucleic acid encoding these molecules) to pancreas tissue (for example by using a pump, or by implantation of a slow release form at the site of the pancreas). The particular mode of administration and the dosage regimen will be selected by the attending clinician, taking into account the particulars of the case (e.g. the subject, the disease, the disease state involved, the particular treatment, and whether the treatment is prophylactic).

Treatment can involve daily or multi-daily or less than daily (such as weekly or monthly etc.) doses over a period of a few days to months, or even years. For example, a therapeutically effective amount of a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be administered in a single dose, twice daily, weekly, or in several doses, for example daily, or during a course of treatment. In a particular non-limiting example, treatment involves once daily dose or twice daily dose.

The amount of mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) administered can be dependent on the subject being treated, the severity of the affliction, and the manner of administration, and can be left to the judgment of the prescribing clinician. Within these bounds, the formulation to be administered will contain a quantity of the mutated FGF2 protein in amounts effective to achieve the desired effect in the subject being treated. A therapeutically effective amount of mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be the amount of the mutant FGF2 protein, or a nucleic acid encoding these molecules that is necessary to treat diabetes, reduce blood glucose levels, and/or treat one or more metabolic diseases (for example a reduction of at least 5%, at least 10%, at least 20%, or at least 50%).

When a viral vector is utilized for administration of an nucleic acid encoding a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), the recipient can receive a dosage of each recombinant virus in the composition in the range of from about 10⁵ to about 10¹⁰ plaque forming units/mg mammal, although a lower or higher dose can be administered. Examples of methods for administering the composition into mammals include, but are not limited to, exposure of cells to the recombinant virus ex vivo, or injection of the composition into the affected tissue or intravenous, subcutaneous, intradermal or intramuscular administration of the virus. Alternatively the recombinant viral vector or combination of recombinant viral vectors may be administered locally by direct injection into the pancreases in a pharmaceutically acceptable carrier. Generally, the quantity of recombinant viral vector, carrying the nucleic acid sequence of the mutated FGF2 protein to be administered (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) is based on the titer of virus particles. An exemplary range to be administered is 10⁵ to 10¹⁰ virus particles per mammal, such as a human.

In some examples, a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), or a nucleic acid encoding the mutated FGF2 protein, is administered in combination (such as sequentially or simultaneously or contemporaneously) with one or more other agents, such as those useful in the treatment of diabetes, insulin resistance, heart disease, dyslipidemia, or combinations thereof.

Anti-diabetic agents are generally categorized into six classes: biguanides; thiazolidinediones; sulfonylureas; inhibitors of carbohydrate absorption; fatty acid oxidase inhibitors and anti-lipolytic drugs; and weight-loss agents. Any of these agents can also be used in the methods disclosed herein. The anti-diabetic agents include those agents disclosed in Diabetes Care, 22(4):623-634. One class of anti-diabetic agents of use is the sulfonylureas, which are believed to increase secretion of insulin, decrease hepatic glucogenesis, and increase insulin receptor sensitivity. Another class of anti-diabetic agents use biguanide antihyperglycemics, which decrease hepatic glucose production and intestinal absorption, and increase peripheral glucose uptake and utilization, without inducing hyperinsulinemia.

In some examples, mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) can be administered in combination with effective doses of anti-diabetic agents (such as biguanides, thiazolidinediones, or incretins), lipid lowering compounds (such as statins or fibrates)). The term “administration in combination” or “co-administration” refers to both concurrent and sequential administration of the active agents. Administration of mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) or a nucleic acid encoding such a mutant FGF2 protein, may also be in combination with lifestyle modifications, such as increased physical activity, low fat diet, low sugar diet, and smoking cessation. Additional agents of use in combination with a mutant FGF2 protein include, without limitation, anti-apoptotic substances such as the Nemo-Binding Domain and compounds that induce proliferation such as cyclin dependent kinase (CDK)-6, CDK-4 and Cyclin D1. Other active agents can be utilized, such as antidiabetic agents for example, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570, aleglitazar, farglitazar, muraglitazar, tesaglitazar, and TZD) and antagonists, PPAR-gamma/alpha modulators (such as KRP 297), alpha-glucosidase inhibitors (e.g., acarbose, voglibose), Dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g., exendin-4) or amylin. In some embodiments the agent is an immunomodulatory factor such as anti-CD3 mAb, growth factors such as HGF, vascular endothelial growth factor (VEGF), platelet derived growth factor (PDGF), lactogens, or parathyroid hormone related protein (PTHrP). In one example, the mutated FGF2 protein is administered in combination with a therapeutically effective amount of another FGF, such as FGF2, heparin, or combinations thereof.

In some embodiments, methods are provided for treating diabetes or pre-diabetes in a subject by administering a therapeutically effective amount of a composition including a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), or a nucleic acid encoding the mutated FGF2 protein, to the subject. The subject can have diabetes type I or diabetes type II. The subject can be any mammalian subject, including human subjects and veterinary subjects such as cats and dogs. The subject can be a child or an adult. The subject can also be administered insulin. The method can include measuring blood glucose levels.

In some examples, the method includes selecting a subject with diabetes, such as type I or type II diabetes, or a subject at risk for diabetes, such as a subject with pre-diabetes. These subjects can be selected for treatment with the disclosed mutated FGF2 proteins (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 , 44, 48, 49, 50, 51, 52, 53, 54, 55, 56, 57, 58, 59, 60, 61, 62, or 63) or nucleic acid molecules encoding such.

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. In other examples, a subject with pre-diabetes may be diagnosed by impaired glucose tolerance (IGT). An OGTT two-hour plasma glucose of greater than or equal to 140 mg/dL and less than 200 mg/dL (7.8-11.0 mM), or a fasting plasma glucose (FPG) concentration of greater than or equal to 100 mg/dL and less than 125 mg/dL (5.6-6.9 mmol/L), or HbA1c levels of greater than or equal to 5.7% and less than 6.4% (5.7-6.4%) is considered to be IGT, and indicates that a subject has pre-diabetes. Additional information can be found in Standards of Medical Care in Diabetes—2010 (American Diabetes Association, Diabetes Care 33:S11-61, 2010).

In some examples, the subject treated with the disclosed compositions and methods has HbA1C of greater than 6.5% or greater than 7%.

In some examples, treating diabetes includes one or more of increasing glucose tolerance, decreasing insulin resistance (for example, decreasing plasma glucose levels, decreasing plasma insulin levels, or a combination thereof), decreasing serum triglycerides, decreasing serum non-HDL lipids (such as one or more of IDL, LDL, or VLDL), decreasing free fatty acid levels, and decreasing HbA1c levels in the subject. In some embodiments, the disclosed methods include measuring glucose tolerance, insulin resistance, plasma glucose levels, plasma insulin levels, serum triglycerides, serum lipids, free fatty acids, and/or HbA1c levels in a subject.

In some examples, administration of a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63) or pre-diabetes, by decreasing of HbA1C, such as a reduction of at least 0.5%, at least 1%, or at least 1.5%, such as a decrease of 0.5% to 0.8%, 0.5% to 1%, 1 to 1.5% or 0.5% to 2%. In some examples the target for HbA1C is less than about 6.5%, such as about 4-6%, 4-6.4%, or 4-6.2%. In some examples, such target levels are achieved within about 26 weeks, within about 40 weeks, or within about 52 weeks. Methods of measuring HbA1C are routine, and the disclosure is not limited to particular methods. Exemplary methods include HPLC, immunoassays, and boronate affinity chromatography.

In some examples, administration of a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), or nucleic acid molecule encoding such, treats diabetes or pre-diabetes by increasing glucose tolerance, for example, by decreasing blood glucose levels (such as two-hour plasma glucose in an OGTT or FPG) in a subject. In some examples, the method includes decreasing blood glucose by at least 5% (such as at least 10%, at least 15%, at least 20%, at least 25%, at least 30%, at least 35%, or more) as compared with a control (such as no administration of any of insulin, or no administration of a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63). In particular examples, a decrease in blood glucose level is determined relative to the starting blood glucose level of the subject (for example, prior to treatment with a mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, o62, or 63), or nucleic acid molecule encoding such). In other examples, decreasing blood glucose levels of a subject includes reduction of blood glucose from a starting point (for example greater than about 126 mg/dL FPG or greater than about 200 mg/dL OGTT two-hour plasma glucose) to a target level (for example, FPG of less than 126 mg/dL or OGTT two-hour plasma glucose of less than 200 mg/dL). In some examples, a target FPG may be less than 100 mg/dL. In other examples, a target OGTT two-hour plasma glucose may be less than 140 mg/dL. Methods to measure blood glucose levels in a subject (for example, in a blood sample from a subject) are routine.

In other embodiments, the disclosed methods include comparing one or more indicator of diabetes (such as glucose tolerance, triglyceride levels, free fatty acid levels, or HbA1c levels) to a control (such as no administration of any of insulin, any mutated FGF2 protein (such as a protein generated using the mutations shown in Table 1, for example in combination with an N-terminal deletion, or a protein having at least 80%, at least 85%, at least 90%, at least 92%, at least 95%, at least 96%, at least 97%, at least 98%, or at least 99% sequence identity to SEQ ID NO: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 59, 60, 61, 62, or 63), or a nucleic acid molecule encoding such), wherein an increase or decrease in the particular indicator relative to the control (as discussed above) indicates effective treatment of diabetes. The control can be any suitable control against which to compare the indicator of diabetes in a subject. In some embodiments, the control is a sample obtained from a healthy subject (such as a subject without diabetes). In some embodiments, the control is a historical control or standard reference value or range of values (such as a previously tested control sample, such as a group of subjects with diabetes, or group of samples from subjects that do not have diabetes and/or a metabolic disorder). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval). In other examples, the control is the subject (or group of subjects) treated with placebo compared to the same subject (or group of subjects) treated with the therapeutic compound in a cross-over study. In further examples, the control is the subject (or group of subjects) prior to treatment.

### EXAMPLE 1

### Preparation of Mutated FGF2 Proteins

Mutated FGF2 proteins can be made using known methods (e.g., see Xia et al., PLoS One. 7(11):e48210, 2012). An example is provided below.

Briefly, a nucleic acid sequence encoding an FGF2 mutant protein (e.g., SEQ ID NO: 8 or a variant thereof) can be fused downstream of an enterokinase (EK) recognition sequence (Asp₄Lys) preceded by a flexible 20 amino acid linker (derived from the S-tag sequence of pBAC-3) and an N-terminal (His)₆ tag. The resulting expressed fusion protein utilizes the (His)₆ tag for efficient purification and can be subsequently processed by EK digestion to yield the mutant FGF2 protein.

The mutant FGF2 protein can be expressed from an E. coli host after induction with isopropyl-β-D-thio-galactoside. The expressed protein can be purified utilizing sequential column chromatography on Ni-nitrilotriacetic acid (NTA) affinity resin followed by ToyoPearl HW-405 size exclusion chromatography. The purified protein can be digested with EK to remove the N-terminal (His)₆ tag, 20 amino acid linker, and (Asp₄Lys) EK recognition sequence. A subsequent second Ni-NTA chromatographic step can be utilized to remove the released N-terminal mutant FGF2 protein (along with any uncleaved fusion protein). Final purification can be performed using HiLoad Superdex 75 size exclusion chromatography equilibrated to 50 mM Na₂PO₄, 100 mM NaCl, 10 mM (NH₄)₂SO₄, 0.1 mM ethylenediaminetetraacetic acid (EDTA), 5 mM L-Methionine, pH at 6.5 (“PBX” buffer); L-Methionine can be included in PBX buffer to limit oxidization of reactive thiols and other potential oxidative degradation.

For storage and use, the purified mutant FGF2 protein can be sterile filtered through a 0.22 micron filter, purged with N₂, snap frozen in dry ice and stored at −80° C. prior to use. The purity of the mutant FGF2 protein can be assessed by both Coomassie Brilliant Blue and Silver Stain Plus (BIO-RAD Laboratories, Inc., Hercules Calif.) stained sodium dodecylsulfate polyacrylamide gel electrophoresis (SDS PAGE). Mutant FGF2 proteins can be prepared in the absence of heparin. Prior to IV bolus, heparin, or PBS, can be added to the protein.

As an alternative to using a tag, mutant FGF2 can be expressed without any tags, and purified using a heparin affinity column and a sepharose column as the final step in the purification. This approach can generate a biologically active peptide.

### EXAMPLE 2

### Mutated FGF2 that Reduces Blood Glucose in ob/ob Mice

We have shown that administration of mature rFGF1 to ob/ob mice can lower blood glucose, but administration of FGF2 has no effect. Based on the crystal structures of FGF-FGFR complexes, point mutations were introduced into FGF2 to increase receptor binding. Such mutant FGF2 proteins can have reduced adverse effects as compared to those observed with thiazolidinediones (TZDs). Similar methods can be used to demonstrate that the other mutant FGF2 proteins provided herein also lower blood glucose.

### Animals

Mice were housed in a temperature-controlled environment with a 12-hour light/12-hour dark cycle and handled according to institutional guidelines complying with U.S. legislation. Male ob/ob mice (B6.V-Lep^{ob}/J, Jackson laboratories) received a standard diet (MI laboratory rodent diet 5001, Harlan Teklad) and acidified water ad libitum. Adiponectin-Cre (B6.FVB-Tg(Adipoq-cre)1Evdr/J, Jackson laboratories) mice were crossed to FGFR1 floxed mice (B6.129S4-Fgfr1tm5.1Sor/J, Jackson laboratories) to generate Adipoq-Cre; FGFR1 fl/fl mice (fat FGFR1KO). Similarly, adiponectin-Cre (B6.FVB-Tg(Adipoq-cre)1Evdr/J, Jackson laboratories) mice were crossed to FGFR2 floxed mice (B6.129X1(Cg)-Fgfr2tm1Dor/J, Jackson laboratories) to generate Adipoq; FGFR2 fl/fl (fat FGFR2KO) mice. FGFR control mice and FGFRKO mice received a high fat diet (high fat (60%) diet F3282, Bio-Serv) for 14 weeks from 6 weeks of age. 0.1 mg/ml solutions in PBS human FGF2 (Prospec, Ness Ziona, Israel), human FGF2 incorporating G19F, H25N and F26Y mutations (mutFGF2; SEQ ID NO: 8), human FGF2 incorporating G19F, H25N, F26Y, and S73Y mutations (SEQ ID NO: 63), and human FGF1 lacking 9 N-terminal amino acids (FGF1ΔNT; SEQ ID NO: 7) were injected subcutaneously into mice.

### Purification of FGF Proteins

Human FGF1 (M1 to D155; SEQ ID NO: 6), N-terminally truncated human FGF1 (FGF1^{ANT}; K25 to D155; SEQ ID NO: 7), human FGF2 incorporating G19F, H25N, F26Y, and S73Y mutations (SEQ ID NO: 63), and mutFGF2 (M1-S155; G19F, H25N, F26Y; SEQ ID NO: 8) were expressed in Escherichia coli cells and purified from the soluble bacterial cell lysate fraction by heparin affinity, ion exchange, and size exclusion chromatographies.

Mice received a standard diet (ob/ob) or high fat diet (C57/BL6 mice, 60% fat, F3282, Bio-Serv) and acidified water ad libitum. Blood glucose levels were monitored either in the ad libitum fed state or following overnight fasting after subcutaneous injection of recombinant FGF1, rFGF1^{ΔNT}, FGF2, or mutFGF2(0.5 mg/kg in PBS).

### Blood Glucose Analysis

Blood was collected by tail bleeding in the ad libitum fed state, and blood glucose levels measured using a OneTouch Ultra glucometer (Lifescan Inc).

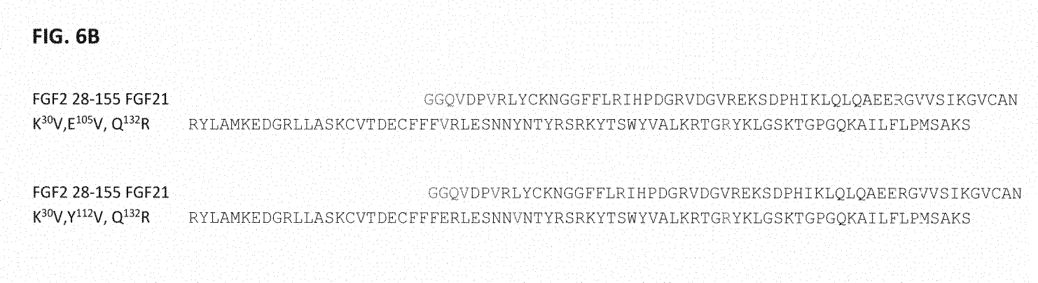

As shown in FIG. 7A, injection of wildtype FGF2 into hyperglycemic mice does not alter their blood glucose levels. In contrast, as shown in FIG. 7B, a mutant FGF2 harboring three point mutations (G19F, H25N, F26Y; SEQ ID NO: 8) acquires the ability to lower blood glucose levels in ob/ob mice 24 hours after injection.

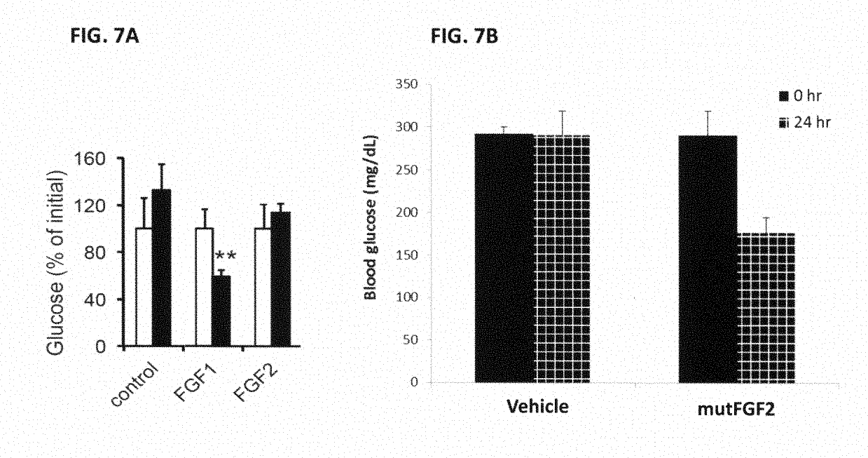

As shown in FIG. 8A, the ability of mutFGF2 (SEQ ID NO :8) to lower blood glucose levels 24 hours after injection is lost in mice lacking the FGFR1 receptor specifically in adipose tissue (fat FGFR1KO mice). As shown in FIG. 8B, the transient reduction in food intake observed with FGF1ΔNT and mutFGF2 is independent of FGFR1 adipose expression.

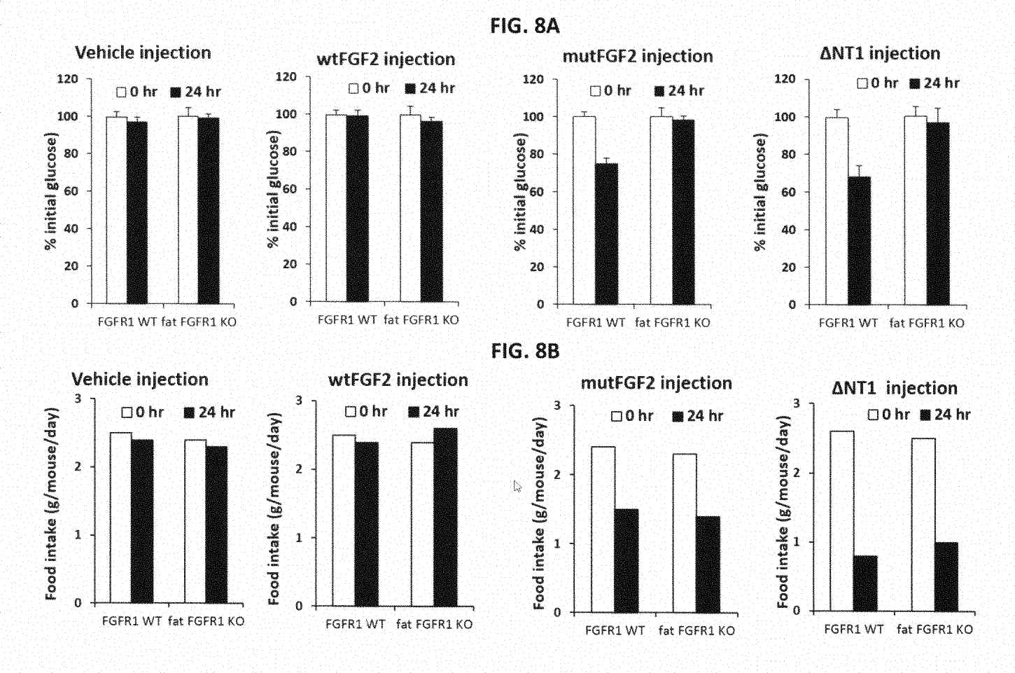

As shown in FIG. 9A, the ability of mutFGF2 (SEQ ID NO :8) to lower blood glucose levels 24 hours after injection is maintained in mice lacking the FGFR2 receptor specifically in adipose tissue (fat FGFR2KO mice). As shown in FIG. 9B, the transient reduction in food intake observed with FGF1ΔNT and mutFGF2 is independent of FGFR2 adipose expression.

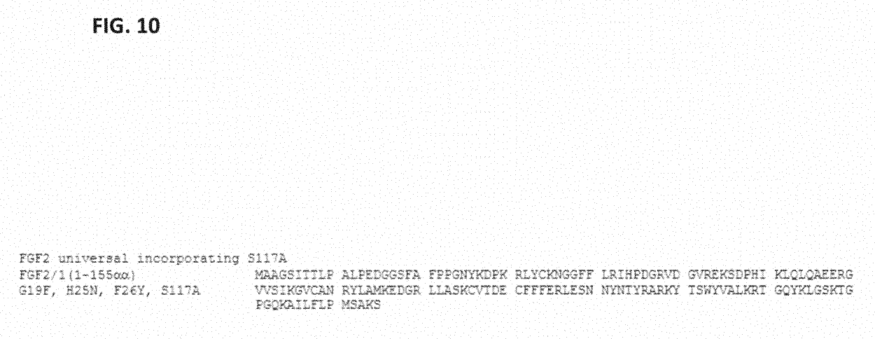

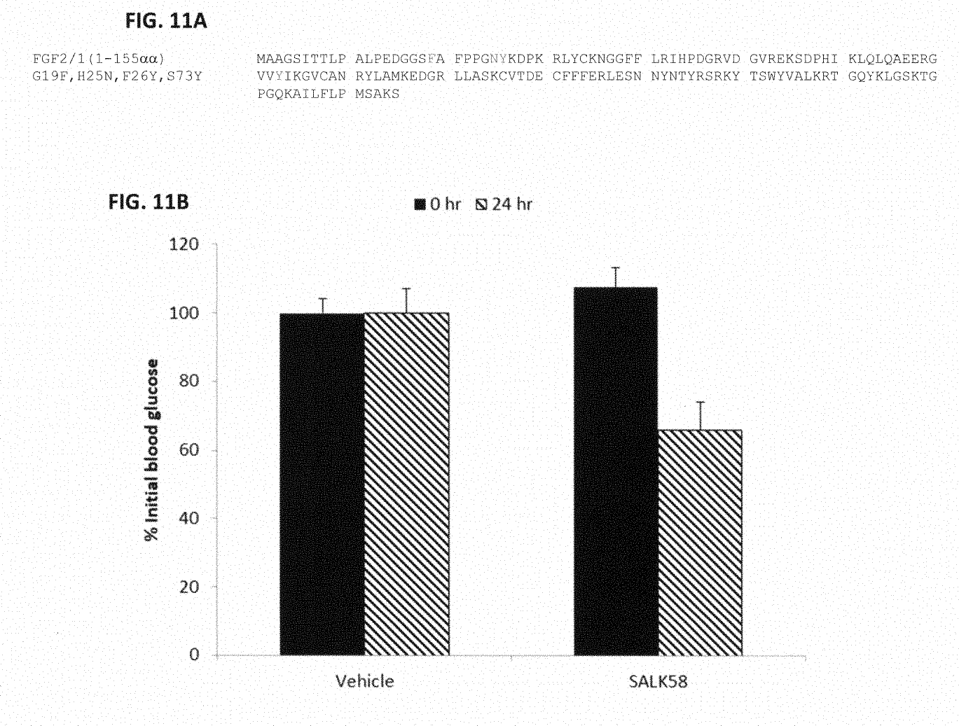

As shown in FIG. 11B, mice injected with a mutant FGF2 (SEQ ID NO: 63, FIG. 11A) acquired glucose lowering abilities in vivo.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. A method of reducing blood glucose in a mammal, comprising:
administering a therapeutically effective amount of a mutated mature fibroblast growth factor (FGF) 2 protein to the mammal, or a nucleic acid molecule encoding the mutated mature FGF2 protein or a vector comprising the nucleic acid molecule, thereby reducing the blood glucose,
wherein the mutated mature FGF2 protein comprises:
a deletion of at least six contiguous N-terminal amino acids;
at least one point mutation;
or combinations thereof.

2. A method of reducing fed and fasting blood glucose, improving insulin sensitivity and glucose tolerance, reducing systemic chronic inflammation, ameliorating hepatic steatosis, or combinations thereof, in a mammal, comprising:
administering a therapeutically effective amount of a mutated mature FGF2 protein to the mammal, or a nucleic acid molecule encoding the mutated FGF2 protein or a vector comprising the nucleic acid molecule, thereby reducing fed and fasting blood glucose, improving insulin sensitivity and glucose tolerance, reducing systemic chronic inflammation, ameliorating hepatic steatosis in a mammal, or combinations thereof, in a mammal,
wherein the mutated mature FGF2 protein comprises:
a deletion of at least six contiguous N-terminal amino acids;
at least one point mutation;
or combinations thereof.

3. A method of treating one or more metabolic diseases in a mammal, comprising:
administering a therapeutically effective amount of a mutated mature fibroblast growth factor (FGF) 2 protein to the mammal, or a nucleic acid molecule encoding the mutated mature FGF2 protein or a vector comprising the nucleic acid molecule, thereby treating the one or more metabolic diseases,
wherein the mutated mature FGF2 protein comprises:
a deletion of at least six contiguous N-terminal amino acids;
at least one point mutation;
or combinations thereof.

4. The method of claim 1, wherein the one or more metabolic diseases is one or more of diabetes, dyslipidemia, polycystic ovary syndrome (PCOS), metabolic syndrome (MetS), obesity, non-alcoholic steatohepatitis (NASH), non-alcoholic fatty liver disease (NAFLD), or hypertension.

5. The method of claim 1, wherein the mutated mature FGF2 protein has reduced mitogenic activity compared to native mature FGF2.

6. The method of claim 1, wherein the therapeutically effective amount of the mutated mature FGF2 protein is at least 0.1 mg/kg.

7. The method of claim 1, wherein the administering is subcutaneous, intraperitoneal, intramuscular, or intravenous.

8. The method of claim 1, wherein the mammal is a cat or dog.

9. The method of claim 1, wherein the mammal is a human.

10. The method of claim 1, wherein the mutated mature FGF2 protein comprises a deletion of at least 9, at least 10, at least 11, at least 12, at least 13, at least 14, at least 15, at least 16, at least 17, at least 18, at least 10, or at least 27 contiguous N-terminal amino acids, wherein the mutated FGF2 protein has reduced mitogenic activity as compared to native mature FGF2 protein.

11. The method of claim 1, wherein the at least one point mutation comprises a mutation at one or more of G19, H25, F26, K30, Y33, R53, Q65, S73, C96, E105, N111, Y112, N113, R116, S117, R118, K119, Y120, T121, S122, W123, K128, R129, Q132, K134, and S137, wherein the numbering refers to the sequence of SEQ ID NO: 3, and wherein the mutated FGF2 protein has reduced mitogenic activity as compared to native mature FGF2 protein.

12. The method of claim 1, wherein the at least one point mutation comprises one or more of the mutations shown in Table 1, wherein the mutated FGF2 protein has reduced mitogenic activity as compared to native mature FGF2 protein.

13. The method of claim 1, wherein the at least one point mutation comprises a mutation at G19, H25, and F26, wherein the numbering refers to the sequence of SEQ ID NO: 3, and wherein the mutated FGF2 protein has reduced mitogenic activity as compared to wild-type mature FGF2 protein.

14. The method of claim 1, wherein the at least one point mutation comprises G19F, H25N, F26Y, wherein the numbering refers to the sequence of SEQ ID NO: 3, and wherein the mutated FGF2 protein has reduced mitogenic activity as compared to wild-type mature FGF2 protein.

15. The method of claim 1, wherein the native mature FGF2 protein comprises SEQ ID NO: 3 or amino acids 10-154 of SEQ ID NO: 5.

16. The method of claim 1, wherein the mutated mature FGF2 protein:
comprises at least 80%, at least 85%, at least 90%, at least 92%, at least 95%, at least 96%, at least 97%, at least 98%, or at least 99% sequence identity to the protein sequence of SEQ ID NO: 44, 48, 49, 50, 51, 52, 53, 54, 55, 56, 57, 58, 59, 60, 61, 62 or 63.;
comprises the protein sequence of SEQ ID NO: 44, 48, 49, 50, 51, 52, 53, 54, 55, 56, 57, 58, 59, 60, 61, 62 or 63; or,
consists of the protein sequence of SEQ ID NO: 44, 48, 49, 50, 51, 52, 53, 54, 55, 56, 57, 58, 59, 60, 61, 62 or 63.

17. An isolated mutated mature fibroblast growth factor (FGF) 2 protein comprising at least 80%, at least 85%, at least 90%, at least 92%, at least 95%, at least 96%, at least 97%, at least 98%, or at least 99% sequence identity to the protein sequence of SEQ ID NO: 44, 48, 49, 50, 51, 52, 53, 54, 55, 56, 57, 58, 59, 60, 61, 62, or 63;
comprising the protein sequence of SEQ ID NO: 44, 48, 49, 50, 51, 52, 53, 54, 55, 56, 57, 58, 59, 60, 61, 62, or 63; or
consisting of the protein sequence of SEQ ID NO: 44, 48, 49, 50, 51, 52, 53, 54, 55, 56, 57, 58, 59, 60, 61, 62, or 63.

18. An isolated nucleic acid molecule encoding the isolated protein of claim 17.

19. A nucleic acid vector comprising the isolated nucleic acid molecule of claim 18.

20. A host cell comprising the vector of claim 19.
